(19) United States
(12) Patent Application Publication
(10) Pub. No.: US 2024/0343739 A1
(43) Pub. Date: Oct. 17, 2024

(54) TWO DIMENSIONAL BENZO[4,5]IMIDAZO[2,1-A]ISOINDOLEINCORPORATED NON-FULLERENE ELECTRON ACCEPTORS FOR ORGANIC PHOTOVOLTAIC DEVICES

(71) Applicant: National Research Council of Canada, Ottawa (CA)

(72) Inventors: Yinghui HE, Ottawa (CA); Jianping LU, Ottawa (CA); Salima ALEM, Ottawa (CA); Ye TAO, Orleans (CA)

(21) Appl. No.: 18/579,671

(22) PCT Filed: Jul. 12, 2022
(86) PCT No.: PCT/CA2022/051080

§ 371 Date: Jan. 16, 2024

(30) Foreign Application Priority Data
Jul. 14, 2021 (CA) 3124916

(51) Int. Cl. C07D 495/22; C08G 61/12; H10K 30/30; H10K 85/60

(52) U.S. Cl. CPC C07D 495/22; C08G 61/12; H10K 30/30; H10K 85/657; C08G 2261/226; C08G 2261/3246; C08G 2261/91

(57) ABSTRACT

The present application provides two dimensional benzo[4,5]imidazo[2,1-a]isoindole incorporated non-fullerene electron acceptors having the structure of Formula I (I), and processes for the synthesis and manufacture thereof. Also provided are semiconductor materials, polymers, oligomers, films and membranes incorporating the non-fullerene acceptor of Formula (I), and the optoelectronic devices made therefrom.

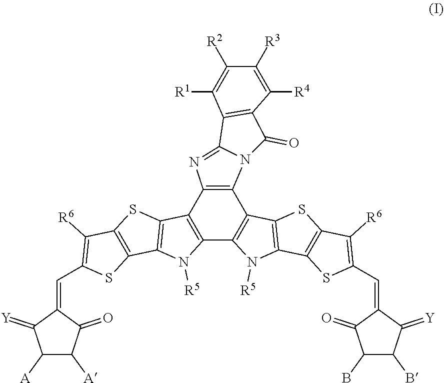

### RELATED APPLICATION

This application claims the priority to Canadian Patent Application No. 3,124,916, filed on 14 Jul. 2021, the contents of which is incorporated herein by reference in its entirety.

### FIELD OF THE INVENTION

The present application pertains to the field of photovoltaic materials and devices. More particularly, the present application relates to non-fullerene electron acceptors, methods of manufacture and uses thereof, and to photovoltaic devices comprising, the non-fullerene electron acceptors.

### INTRODUCTION

Organic photovoltaics (OPVs) are currently considered to be one of the most promising photovoltaic technologies due to their light weight, mechanical robustness, and high throughput production by solution processes¹⁻⁵. This technology uses organic materials to convert light into electricity by pairing electron donors with electron acceptors, which are sandwiched between the anode and the cathode of the organic photovoltaic device. While electron donors have been well developed over the years, electron acceptors are relatively limited. Most commercial electron acceptors are fullerene-based.

In recent years, the field of OPVs has experienced rapid development with the power conversion efficiencies (PCEs) now reaching 18% for single-junction devices under one-sun irradiation⁶⁻¹⁰. This significant advancement was mainly driven by the emergence of a series of high-performance non-fullerene acceptors (NFAs), which feature a combination of a ladder-type electron-deficient central fused core, electron-donating bridges, and two electron-accepting end-groups in a C₂ symmetric manner¹¹⁻¹³. These high performance NFAs can provide complementary absorption to their donor counterparts and require less driving force for charge carrier generation as compared with fullerene aceptors¹⁴⁻¹⁶, thereby simultaneously increasing the short-circuit current (J_{sc}) and minimizing the voltage loss.

Despite the recent success, the PCEs of OPVs still lag behind their inorganic semiconductors and perovskite counterparts that have already demonstrated PCE over 25%¹⁷. While the J_{SC} and fill factor (FF) of OPVs are already comparable with those of perovskite solar cells, the open-circuit voltage (V_{OC}) of OPVs is still significantly lower (Table 1). The relatively low V_{OC} of OPVs has become the main hindrance to further improvement of the device PCE. Currently, most of the high-performance OPVs are only showing V_{OC} between 0.8 and 0.9 V^{10,15}. With the light absorber's optical bandgap (E_{g}) typically in the range of 1.3 eV to 1.7 eV, there is a need to improve the V_{OC} and thus boost overall device performance.

TABLE 1
A comparison of state-of the-art devices
for OPVs and perovskite solar cells

| Device | J_{SC} (mA/cm²) | V_{OC} (V) | FF | PCE (%) | Ref |
|---|---|---|---|---|---|
| OPV1 | 27.70 | 0.859 | 0.766 | 18.2 | 7 |
| OPV2 | 26.20 | 0.839 | 0.811 | 17.8 | 6 |
| Perovskite | 25.09 | 1.194 | 0.847 | 25.4 | 17 |

Studies have shown that the V_{OC} of OPVs is strongly related to the energy of the charge-transfer (CT) state of the donor-acceptor in the bulk heterojunction (see, FIG. 1)^{18,19}. Because a driving force is needed to break up the exciton, the energy of CT state is always lower than the E_{g} of the lower bandgap component in the blend, which means there is an energy loss. Reducing the offsets between the LUMO levels of the donor and acceptor is a frequently used strategy to minimize the energy loss associated with CT state. With the use of the NFA Y6 in OPVs, researchers have been able to reduce the required donor-acceptor LUMO energy offsets and therefore increase the V_{OC} of OPVs.

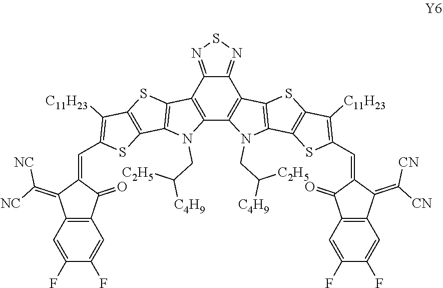

Although the donor-acceptor LUMO energy offsets have been minimized with the use of the NFA Y6, there is still a significant energy gap (0.4-0.6 eV²¹) between the V_{OC} and the E_{g} in OPVs. In addition to the energy loss caused by CT state, the relatively high radiative recombination loss due to absorption edge broadening effects and the strong non-radiative recombination loss due to energetic disorders are also a main cause for the energy loss in OPVs^{22,23}. To date, there has been little work done to study or tackle this aspect of energy loss. In addition, electric circuits usually require certain voltages to operate. Higher voltage output from an individual PV cell would mean fewer cells to be connected in series, which would simplify the fabrication process, but is not possible using Y6. Further, the solubility of Y6 is not sufficient for use in large-area device fabrication using scalable processing techniques, such as blade coating, slot-die coating, flexographic printing and gravure printing.

There has been a growing interest in the potential application of OPVs under indoor lighting conditions, since the efficiency of OPVs greatly improves at low light intensity²⁴⁻²⁷ The fast growth of internet of things (IoT) has created a large demand for off-grid electronic devices such as sensors and Bluetooth devices. Indoor light harvesting OPVs can be a good candidate as the off-grid power source since they offer advantages of light weight and compatibility with roll-to-roll process²⁸⁻³⁰. However, current state-of-the-art OPV materials may not be ideal for indoor lighting conditions, because the design rules of photoactive materials for indoor OPVs often differ from those of outdoor OPVs. For example, the indoor lighting emission is mainly in the range from 400 to 700 nm³¹, while cutting-edge NFAs, such as Y6¹⁵, were designed to focus on the range from 750 nm to 900 nm. This spectral mismatch can lead to poor indoor light harvesting efficiency. Therefore, the absorption spectra of NFAs need to be tuned accordingly in order to develop highly efficient indoor OPV cells.

Using current NFAs, it would be necessary to connect quite a few OPV cells together in series in order to obtain enough output voltages from the OPV panels to drive external sensors or electric circuits. This will increase OPV panel production complexity and reduce the overall production yields.

There remains a need for alterative NFAs to address the drawbacks associated with currently available NFAs.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide two dimensional benzo[4,5]imidazo[2,1-a]isoindole incorporated non-fullerene electron acceptors for organic photovoltaic devices. In accordance with an aspect of the present application, there is provided a non-fullerene acceptor compound of Formula I, referred to herein as a BIID-based non-fullerene acceptor compound due to the incorporation of the electron-withdrawing core, which is 5a,9a-dihydro-11H-benzo[4,5]imidazo[2,1-a]isoindol-11-one (BIID),

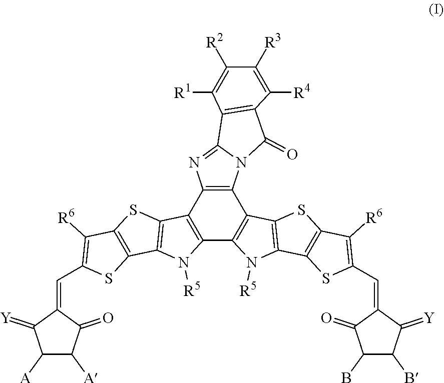

wherein:
R¹, R², R³ and R⁴ are the same or different and each is independently F, H, Cl, Br, I, a substituted or unsubstituted C₁-C₁₂-alkyl, a substituted or unsubstituted C₁-C₁₂-alkoxy, a substituted or unsubstituted aryl group or a substituted or unsubstituted heteroaryl group, or optionally one of R¹-R⁴ is a C₁-C₆ alkyl, such as a methyl, ethyl, propyl, or butyl, and the other three are H;
each R⁶ is independently a substituted or unsubstituted C₁-C₃₀-alkyl group, which is optionally a branched C₆-C₂₀ alkyl, such as a branched C₈₋₁₂-alkyl;
each R⁶ is independently a substituted or unsubstituted C₁-C₃₀-alkyl group, which is optionally a C₆-C₂₀ alkyl, such as a linear C₁ alkyl;
each Y is independently C(CN)₂ or O, preferably C(CN)₂;
A and A′ together with the carbons to which they are attached form an aromatic or heteraromatic ring; and
B and B′ together with the carbons to which they are attached form an aromatic or heteraromatic ring.

Optionally, in the non-fullerene acceptor compound of formula I, A and A′ together with the carbons to which they are attached form an aromatic or heteraromatic moiety that is:

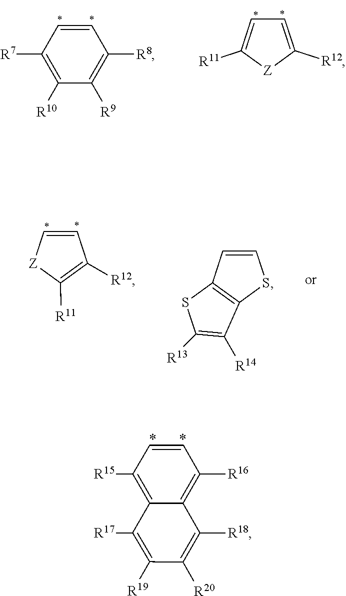

where the asterisks show the point of attachment;
and/or
B and B′ together with the carbons to which they are attached form an aromatic or heteraromatic ring, for example, B and B′ together with the carbons to which they are attached form an aromatic or heteraromatic moiety that is:

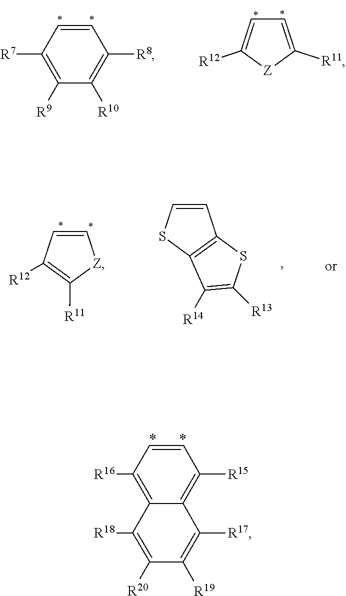

where the asterisks show the point of attachment;
wherein:
R⁷, R⁸, R⁹ and R¹⁰ are each the same or different and are independently F, H, Cl, Br, I, a C₁-C₁₂-alkyl or a C₁-C₁₂-alkoxy group;
Z is O, S, Se, or NR, where R is a C₁-C₁₂-alkyl; and
R¹¹ to R²⁰ are each the same or different and are independently F, H, Cl, Br, I, a C₁-C₁₂-alkyl or a C₁-C₁₂-alkoxy group.

In accordance with one embodiment, there is provided a BIID-based non-fullerene acceptor compound having the structure of Formula (II), Formula (III), Formula (IV), Formula (V) or Formula (VI):

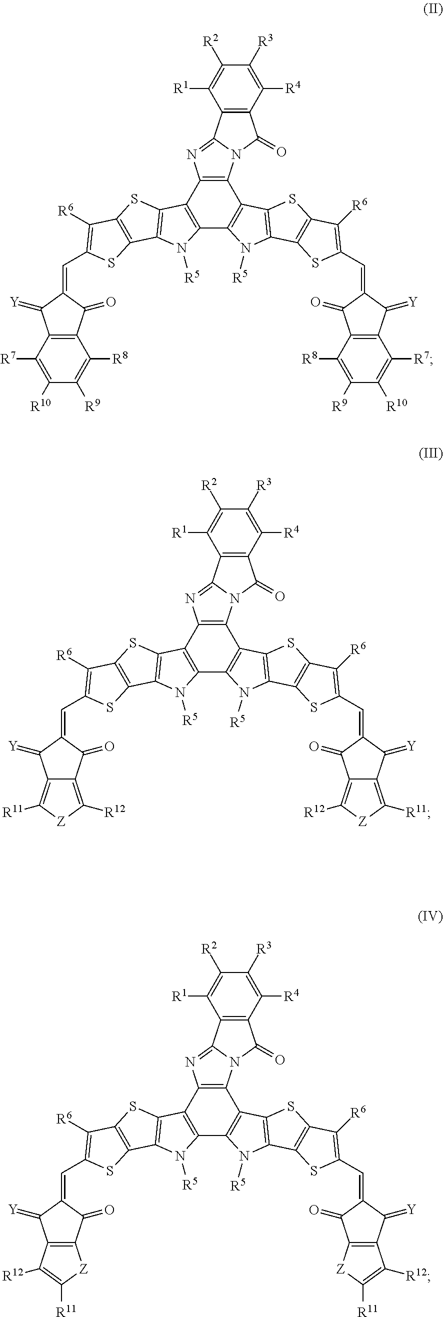

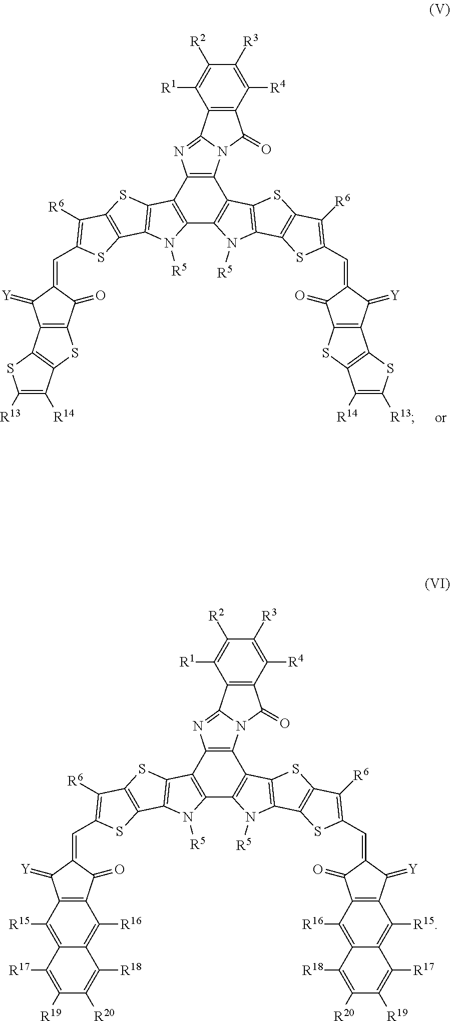

In accordance with some embodiments, there is provided a compound of Formula II, wherein R⁷ and R⁸ are both H and R⁹ and R¹⁰ are each independently H, Cl or F. In some examples, each R⁹ and R¹⁰ are the same and are either Cl or F, preferably F.

In accordance with another aspect, there is provided a semiconductor material (e.g., a bulk heterojunction organic material) comprising a BIID-based non-fullerene acceptor compound as defined herein in combination with an electron donor polymer. Optionally the polymer donor is a middle bandgap donor polymer, such as, but not limited to, PTQ10, J52, and PCDTBT. In a specific embodiment PBDB-T-2F (“PM6”) is employed as the donor polymer.

In accordance with another aspect, there is provided a polymer or an oligomer comprising a BIID-based non-fullerene acceptor compound according as described herein copolymerized with an electron-donating co-monomer or an electron-withdrawing co-monomer. In some examples, the polymer or oligomer has a ratio of electron-accepting monomer to electron-donating or electron-withdrawing co-monomer in a range of from 1:99 to 99:1 mol % and/or comprises from 2 to 20,000 or from 2 to 10,000 monomeric units

In accordance with some embodiments, the polymer or oligomer is made from one or more BIID-based NFA monomers in combination with one or more electron-donating co-monomer, which is optionally one or more of a substituted or unsubstituted phenyl, thiophene, fluorene, carbazole, benzodithiophene, pyrrole, indenofluorene, indolocarbazole, dibenzosilole, dithienosilole, benzo[1,2-b;3,4-b]dithiophene, benzo[2,1-b:3,4-b′]dithiophene, cyclopenta[2,1-b:3,4-b′]dithiophene, thieno[3,2-b]thiophene, thieno[3,4-b]thiophene or dithieno[3,2-b:2′,3′-d]pyrrole.

In accordance with other embodiments, the polymer or oligomer is made from one or more BIID-based NFA monomers in combination with one or more electron-withdrawing co-monomer, which is optionally one or more of 2,1,3-benzothiadiazole, 2H-benzo[d][1,2,3]triazole, benzo[c][1,2,5]oxadiazole, benzo[c][1,2,5]selenadiazole, diketopyrrolo[3,4-c]pyrrole-1,4-dione, ester or ketone substituted thieno[3,4-b]thiophene, thieno[3,4-c]pyrrole-4,6-dione, isoindigo, or quinoxaline.

In accordance with another aspect, there is provided a film or membrane comprising a BIID-based NFA compound as described herein, or a semiconductor material, polymer or oligomer made from a BIID-based NFA compound as described herein.

In accordance with another aspect, there is provided an optoelectronic device comprising a BIID-based NFA compound as described herein, or a film or membrane comprising a BIID-based NFA compound as described herein, or a semiconductor material, polymer or oligomer made from a BIID-based NFA compound as described herein. The optoelectronic device is can be an organic photovoltaic cell or device, an electroluminescence device, a field effect transistor, an optical sensor, or a thermoelectric device.

In accordance with another aspect, there is provided a process for synthesizing a compound of Formula (I), comprising the steps of:
a. reducing the compound of Formula (VII), optionally with LiAlH₄,

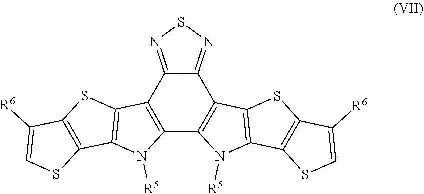

to produce the compound of Formula (VIII)

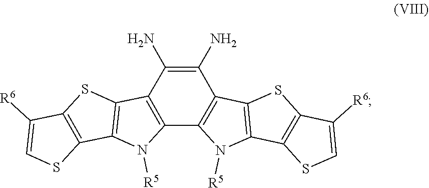

b. treating the compound of Formula (VIII) with a phthalic hydride of Formula (IX)

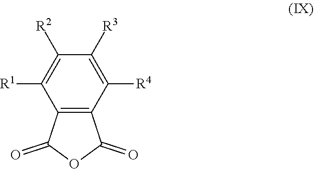

to form a compound of Formula (X)

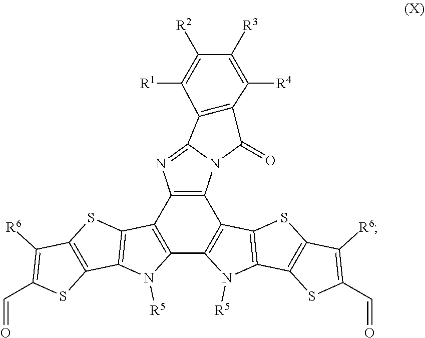

and
c. reacting the compound of Formula (X) with a compound of Formula (XI) and or a compound of Formula (XII)

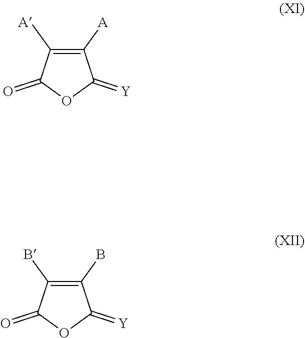

to produce the compound of Formula (I), wherein R¹-R⁶, A, A′, B, B′ and Y are as defined above.

### BRIEF DESCRIPTION OF THE FIGURES

For a better understanding of the application as described herein, as well as other aspects and further features thereof, reference is made to the following description which is to be used in conjunction with the accompanying drawings, where:

FIG. 1 is an illustration of the charge transfer state of a donor-acceptor in bulk heterojunction²⁰;

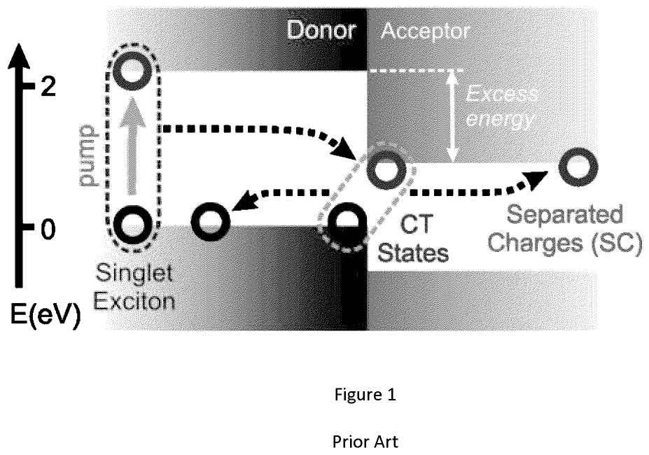

FIG. 2 is a schematic of the 5a,9a-dihydro-11H-benzo[4,5]imidazo[2,1-a]isoindol-11-one (BIID)-based non-fullerene acceptor illustrating regions of the molecule;

FIG. 3 depicts UV-vis absorption spectra of BIID2 in chloroform solution and in film;

FIG. 4 depicts cyclic voltammograms of BIID2;

FIG. 5 depicts the current density-Voltage (J-V) characteristics (a) and external quantum efficiency (EQE) spectrum (b) of an OPV device based on the BIID2:PM6 blend.

FIG. 6 depicts UV-vis absorption spectra of BIID3 in solution and in a thin film;

FIG. 7 depicts cyclic voltammograms of BIID3;

FIG. 8 depicts (a) the J-V curves for OPV devices containing PM6:Y6 and PM6:BIID3 under one sun illumination; (b) the EQE spectra for the two OPV devices; and (c) the J-V curves for the two OPV devices under a 1300 Lux LED illumination; and

FIG. 9 graphically depicts (a) the dependence of J_{sc} on light intensity; and (b) the dependence of V_{OC} on light intensity for BIID3 and Y6-containing devices.

### DETAILED DESCRIPTION

### Definitions

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or ingredient(s) as appropriate.

Reference throughout this specification to “one embodiment,” “an embodiment,” “another embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “substituted” refers to at least one hydrogen atom of a functional group being replaced with a non-hydrogen group, provided that normal valencies are maintained and that the substitution results in a stable compound. When a group is noted as being “substituted”, the substituents are selected from the exemplary group including, but not limited to, halo (e.g., chloro, fluoro or bromo), oxy, carboxy, hydroxy, amino, amido, nitro, thio, C₁-C₃₀-alkyl, C₂-C₃₀-alkenyl, C₂-C₃₀-alkynyl, C₆-C₃₀-aryl, C₆-C₃₀-heteroaryl having one or more N, O or S in the ring, C₇-C₃₆-alkaryl, C₁-C₃₀-alkoxy, C₂-C₃₀-alkenoxy, C₂-C₃₀-alkynoxy, C₆-C₃₀-aryloxy, C₁-C₃₀-alkylamino, C₂-C₆₀-dialkylamino, C₁-C₃₀-alkamido, C₂-C₃₀-carboxy or C₁-C₃₀-carbonyl, and mixtures thereof and the like. In some embodiments, the substituents are selected from the group halo (e.g., chloro, fluoro or bromo), oxy, carboxy, hydroxy, nitro, thio, C₁-C₂₀-alkyl, C₂-C₂₀-alkenyl, C₂-C₂₀-alkynyl, C₆-C₂₀-aryl, C₆-C₂₀-heteroaryl having one or more N, O or S in the ring, C₇-C₂₄-alkaryl, C₁-C₂₀-alkoxy, C₂-C₂₀-alkenoxy, C₂-C₂₀-alkynoxy, C₆-C₂₀-aryloxy, C₂-C₄₀-dialkylamino, C₂-C₂₀-carboxy or C₁-C₂₀-carbonyl, and mixtures thereof.

As used herein, the term “alkyl,” unless otherwise specified, is intended to have its accustomed meaning of a straight or branched chain, saturated hydrocarbon, for example, methyl, ethyl, n-propyl, isopropyl, n-butyl, sec-butyl, t-butyl, n-pentyl, sec-pentyl, t-pentyl, neopentyl, and the like. In some embodiments, alkyl groups have from 1 to 30 carbon atoms, or 1 to 20 carbon atoms, or from 1 to 12 carbon atoms, or from 1 to 8 carbon atoms, or from 1 to 6 carbon atoms. As used herein, the term “C₂-C₃₀ alkyl” refers to an alkyl group, as defined above, containing at least 2, and at most 30, carbon atoms. The term “cycloalkyl” as used herein, is also intended to have its accustomed meaning of a cyclic, saturated hydrocarbon, such as, for example, cyclopropyl, cyclobutyl, cyclopentyl, cyclohexyl, cycloheptyl, cyclooctyl, or the like. In some embodiments, cycloalkyl groups have from 3 to 10 carbon atoms, or from 3 to 8 carbon atoms, or from 3 to 6 carbon atoms, or 5 or 6 carbon atoms. A “substituted alkyl” or “substituted cycloalkyl” includes one or more substituent, as defined above. Preferably, a “substituted alkyl” or “substituted cycloalkyl” includes one or two substituents, as defined above.

As used herein, the term “alkenyl” refers to a hydrocarbon group, e.g., from 2 to 30 carbon atoms, or from 2 to 20 carbon atoms, or from 2 to 12 carbon atoms, and having at least one carbon-carbon double bond. Non-limiting examples of “alkenyl”, as used herein include, vinyl (ethenyl), propenyl, 2-methyl-1-propenyl, 1-butenyl, 2-butenyl, and isobutenyl. As used herein, the term “C₂-C₃₀ alkenyl” refers to an alkenyl group, as defined above, containing at least 2, and at most 30, carbon atoms.

As used herein, the term “alkynyl” refers to a hydrocarbon group, e.g., from 2 to 30 atoms, or from 2 to 20 carbon atoms, or from 2 to 12 carbon atoms, and having at least one carbon-carbon triple bond. Non-limiting examples of “alkynyl”, as used herein, include but are not limited to ethynyl (acetylenyl), 1-propynyl, 1-butynyl, 2-butynyl, 1-pentynyl, and 1-hexynyl. As used herein, the term “C₂-C_{3M} alkynyl” refers to an alkynyl group, as defined above, containing at least 2, and at most 30, carbon atoms.

As used herein, the term “alkoxy” refers to the group RₐO—, where Rₐ is alkyl as defined above and the term “C₁-C₁₂ alkoxy” refers to the group RₐO—, where Rₐ is C₁-C₁₂ alkyl as defined above. Non-limiting examples of “alkoxy” are methoxy, ethoxy, propyloxy, and isopropyloxy.

As used herein, the term “aryl,” unless otherwise specified, is intended to mean an aromatic hydrocarbon system, for example, phenyl, naphthyl, phenanthrenyl, anthracenyl, pyrenyl, and the like. Included within the term “aryl” are heteroaryl groups including one or more heteroatom (e.g., N, O or S), preferably 1 to 3 heteroatoms, in the aromatic system. In some embodiments, aryl or heteroaryl groups have from 6 to 30 carbon atoms, or from 6 to 18 carbon atoms, or from 6 to 14 carbon atoms, or from 6 to 10 carbon atoms. Non-limiting examples of aryl include phenyl, biphenyl, naphthyl and anthracyl and non-limiting examples of the heteroaryl groups include pyridinyl, pyridazinyl, pyrimidyl, pyrazyl, triazinyl, pyrrolyl, pyrazolyl, imidazolyl, (1,2,3,)-triazolyl, (1,2,4)-triazolyl, pyrazinyl, pyrimidinyl, tetrazolyl, furyl, thienyl, isoxazolyl, thiazolyl, isoxazolyl, oxazolyl, benzofuranyl, benzothiophenyl, indolyl, 1H-indazolyl, indolinyl, benzopyrazolyl, 1,3-benzodioxolyl, benzoxazolyl, quinolinyl, isoquinolinyl, benzimidazolyl, quinazolinyl, pyrido[2,3-b]pyrazinyl, pyrido[3,2-c]pyridazinyl, pyrido[3,4-b]-pyridinyl, quinoxalinyl, 1,4-benzisoxazinyl, and benzothiazolyl. A “substituted aryl” or “substituted heteroaryl” includes one or more substituent, as defined above. Preferably, a “substituted aryl” or “substituted heteroaryl” includes one or two substituents, as defined above.

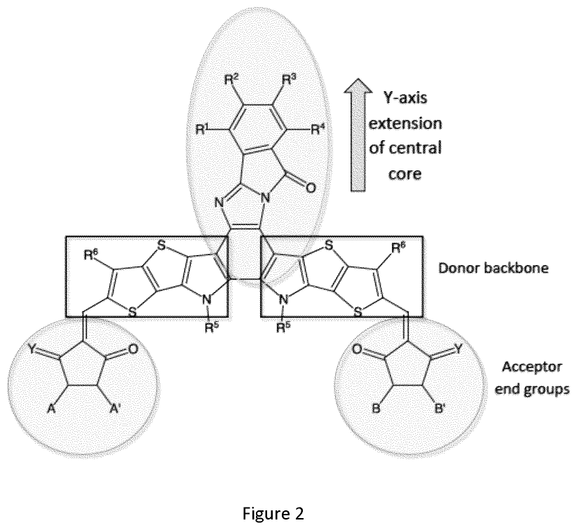

The present inventors have developed a series of non-fullerene acceptors (NFAs) based on the electron-withdrawing core, 5a,9a-dihydro-11H-benzo[4,5]imidazo[2,1-a]isoindol-11-one (BIID). With reference to FIG. 2, these BIID-based NFAs were developed as alternatives to the Y6 NFA. In comparison to Y6, the central core portion of the NFA compounds of the present invention were extended in the y-axis by incorporation of the rigid, two-dimensional, electron-withdrawing core, BIID. The incorporation of BIID can promote intermolecular interaction and, thereby, improve packing of the NFA molecules in their solid state. Surprisingly, the extension of the central core portion with BIID did not negatively affect the solubility of the molecule. Consequently, this extension of the central core portion by incorporation of BIID, contributed to a significantly higher V_{OC} (in comparison to Y6), while maintaining good PCE %. The asymmetry introduced by incorporation of BIID also contributed to the higher V_{oc}.

In addition, π extension in the acceptor end groups of the BIID-based NFA compound of the present invention can improve optical absorption and/or improve π-π stacking to enhance film ordering and carrier mobility. The optional incorporation of halogens, for example, fluorines, in the acceptor end groups of these NFA compounds can be used to further suppress charge recombination loss.

The BIID-based NFA compounds of the present application have a larger π-conjugation than Y6 and include versatile functional groups that are suitable for chemical modification in order to further optimize the NFA for different applications and/or for improved performance. For example, tunability of the BIID-based NFA compounds is further achieved by incorporating different side chain moieties and functional groups in the central core. In contrast, similar modifications cannot be incorporated in Y6 because of the lack of reaction sites.

The present BIID-based NFAs perform well in OPVs. Without wishing to be bound by theory, this is credited to the high V_{OC} obtained, which benefits mainly from the suppressed trap-assisted recombination. The BIID core-based molecular design allows further electronic property tuning, precise morphology optimization, and solution processability, for example, for the use in next-step high performance indoor OPVs.

The present application provides BIID-based NFA compounds having the general chemical structure of Formula I

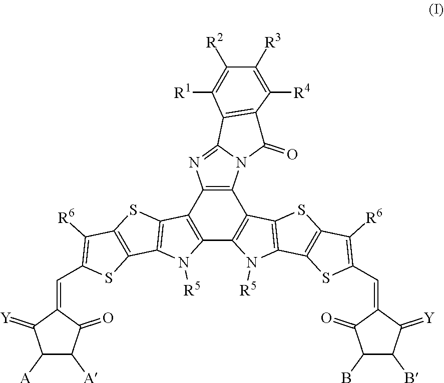

wherein:
R¹, R², R³ and R⁴ are the same or different and each is independently F, H, Cl, Br, I, a substituted or unsubstituted C₁-C₁₂-alkyl, a substituted or unsubstituted C₁-C₁₂-alkoxy, a substituted or unsubstituted aryl group or a substituted or unsubstituted heteroaryl group;
each R⁵ is independently a substituted or unsubstituted C₁-C₃₀-alkyl group;
each R⁶ is independently a substituted or unsubstituted C₁-C₃₀-alkyl group;
each Y is independently C(CN)₂ or O;
A and A′ together with the carbons to which they are attached form an aromatic or heteraromatic ring, for example, A and A′ together with the carbons to which they are attached form an aromatic or heteraromatic moiety that is:

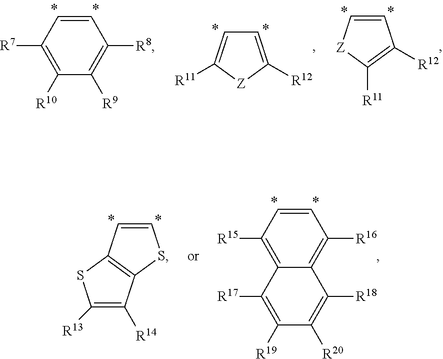

where the asterisks show the point of attachment;
B and B′ together with the carbons to which they are attached form an aromatic or heteraromatic ring, for example, B and B′ together with the carbons to which they are attached form an aromatic or heteraromatic moiety that is:

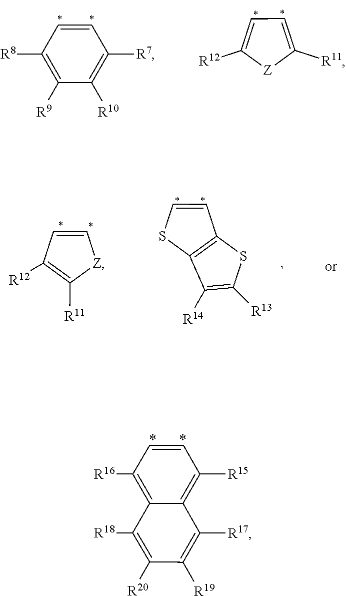

where the asterisks show the point of attachment;
wherein:
R⁷, R⁸, R⁹ and R¹⁰ are each the same or different and are independently F, H, Cl, Br, I, a C₁-C₁₂-alkyl or a C₁-C₁₂-alkoxy group;
Z is O, S, Se, or NR, where R is a C₁-C₁₂-alkyl; and
R¹¹ to R²⁰ are each the same or different and are independently F, H, Cl, Br, I, a C₁-C₁₂-alkyl or a C₁-C₁₂-alkoxy group.

In a specific embodiment there is provided a compound of Formula (I), wherein Y is C(CN)₂. In some examples, R⁵ is a branched C₆-C₂₀ alkyl, for example, a branched C₈-alkyl or a branched C₁₂alkyl, and R⁶ is a C₆-C₂₀ alkyl, such as a linear C₁₁ alkyl.

In some embodiments of the compound of Formula (I), one of R¹-R⁴ is a C₁-C₆ alkyl, such as a methyl, ethyl, propyl, or butyl, and the other three are H. Optionally, R³ is a t-butyl.

In accordance with some embodiments of the present application, the BIID-based NFA compound has the structure of Formula II:

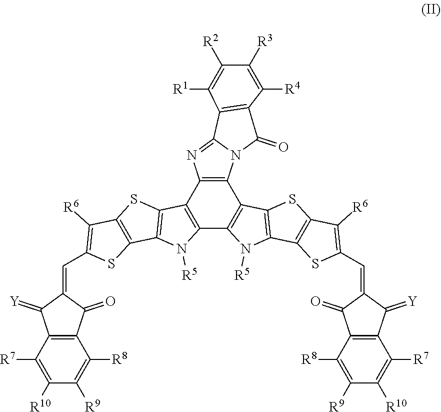

wherein R¹-R¹⁰ and Y are as defined above.

In a specific embodiment there is provided a compound of Formula (II), wherein Y is C(CN)₂. In some examples, R⁵ is a branched C₆-C₂₀ alkyl, for example, a branched C₈-alkyl or a branched C₁₂alkyl, and R⁶ is a C₆-C₂₀ alkyl, such as a linear C₁ alkyl.

In some embodiments of the compound of Formula (II), one of R¹-R⁴ is a C₁-C₆ alkyl, such as a methyl, ethyl, propyl, or butyl, and the other three are H. Optionally, R³ is a t-butyl.

In some embodiments of the compound of Formula (II), the R⁷ and R⁸ groups are H and the R⁹ and R¹⁰ groups are each independently H, Cl or F, or all of the R⁹ and R¹⁰ groups are the same and are H, Cl or F.

In accordance with some embodiments of the present application, the BIID-based NFA compound has the structure of Formula (III):

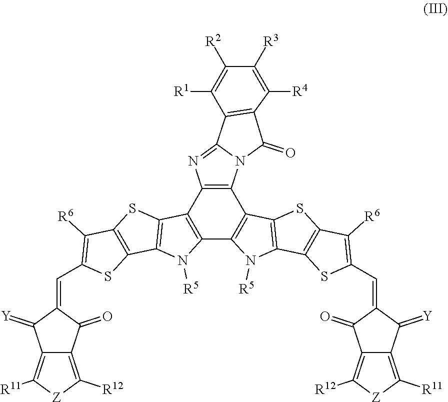

In a specific embodiment there is provided a compound of Formula (III), wherein Y is C(CN)₂. In some examples, R⁵ is a branched C₆-C₂₀ alkyl, for example, a branched C₈-alkyl or a branched C₁₂alkyl, and R⁶ is a C₆-C₂₀ alkyl, such as a linear C₁ alkyl.

In some embodiments of the compound of Formula (III), one of R¹-R⁴ is a C₁-C₆ alkyl, such as a methyl, ethyl, propyl, or butyl, and the other three are H. Optionally, R³ is a t-butyl. Optionally, each of R¹¹ and R¹² are H.

In accordance with some embodiments of the present application, the BIID-based NFA compound has the structure of Formula (IV):

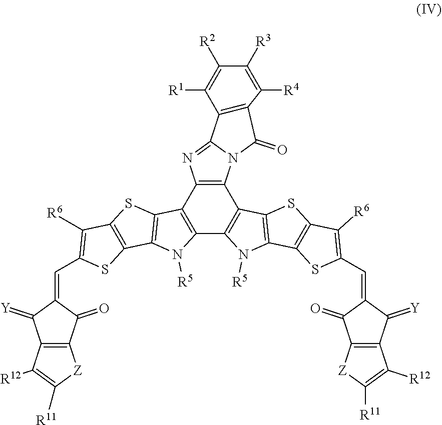

In a specific embodiment there is provided a compound of Formula (IV), wherein Y is C(CN)₂. In some examples, R⁵ is a branched C₆-C₂₀ alkyl, for example, a branched C₈-alkyl or a branched C₂alkyl, and R⁶ is a C₆-C₂₀ alkyl, such as a linear C₁ alkyl.

In some embodiments of the compound of Formula (IV), one of R¹-R⁴ is a C₁-C₆ alkyl, such as a methyl, ethyl, propyl, or butyl, and the other three are H. Optionally, R³ is a t-butyl. In some embodiments, R¹² is H or a C₁-C₈ alkyl, and R¹¹ is F.

In accordance with some embodiments of the present application, the BIID-based NFA compound has the structure of Formula (V):

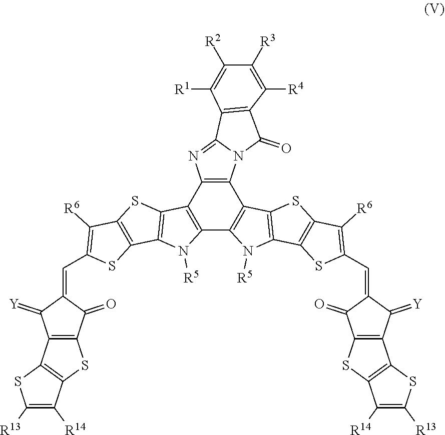

In a specific embodiment there is provided a compound of Formula (V), wherein Y is C(CN)₂. In some examples, R⁵ is a branched C₆-C₂₀ alkyl, for example, a branched C₈-alkyl or a branched C₁₂ alkyl, and R⁶ is a C₆-C₂₀ alkyl, such as a linear C₁ alkyl.

In some embodiments of the compound of Formula (V), one of R¹-R⁴ is a C₁-C₆ alkyl, such as a methyl, ethyl, propyl, or butyl, and the other three are H. Optionally, R³ is a t-butyl. In some embodiments R¹⁴ is H or a C₁-C₈ alkyl, and R¹³ is F.

In accordance with some embodiments of the present application, the BIID-based NFA compound has the structure of Formula (VI):

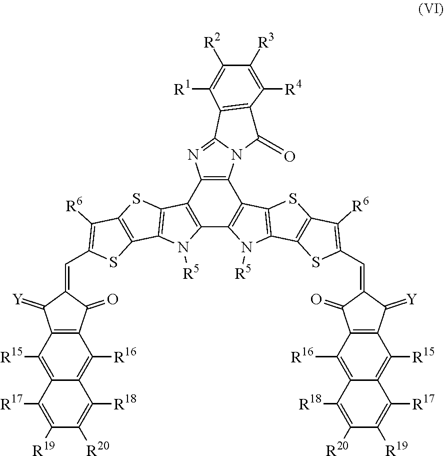

In a specific embodiment there is provided a compound of Formula (VI), wherein Y is C(CN)₂. In some examples, R⁵ is a branched C₆-C₂₀ alkyl, for example, a branched C₈-alkyl or a branched C₁₂ alkyl, and R⁶ is a C₆-C₂₀ alkyl, such as a linear C₁ alkyl.

In some embodiments of the compound of Formula (VI), one of R¹-R⁴ is a C₁-C₆ alkyl, such as a methyl, ethyl, propyl, or butyl, and the other three are H. Optionally, R³ is a t-butyl. In some embodiments, each of R15-R20 are independently H or a C₁-C₁₂ alkyl.

Certain of the compounds described herein may contain one or more chiral atoms, or may otherwise be capable of existing as two enantiomers. The compounds of this application include mixtures of enantiomers as well as purified enantiomers or enantiomerically enriched mixtures. Also provided herein are the individual isomers of the compounds represented by formula (I) above as well as any wholly or partially equilibrated mixtures thereof. The present application also covers the individual isomers of the compounds represented by the formulas above as mixtures with isomers thereof in which one or more chiral centers are inverted.

The presence of a double bond is possible in the compounds described herein, accordingly also included in the present BIID-based NFA compounds are their respective pure E and Z geometric isomers as well as mixtures of E and Z isomers, without any limiting ratios set on prevalence of Z to E isomers.

### Synthesis

The present BIID-based NFA compounds can be prepared using various synthetic methods. Provided herein is a process for synthesis of an embodiment of the compound of Formula I (in which B is the same as A, and B′ is the same and A′) according to the reactions shown in Scheme I:

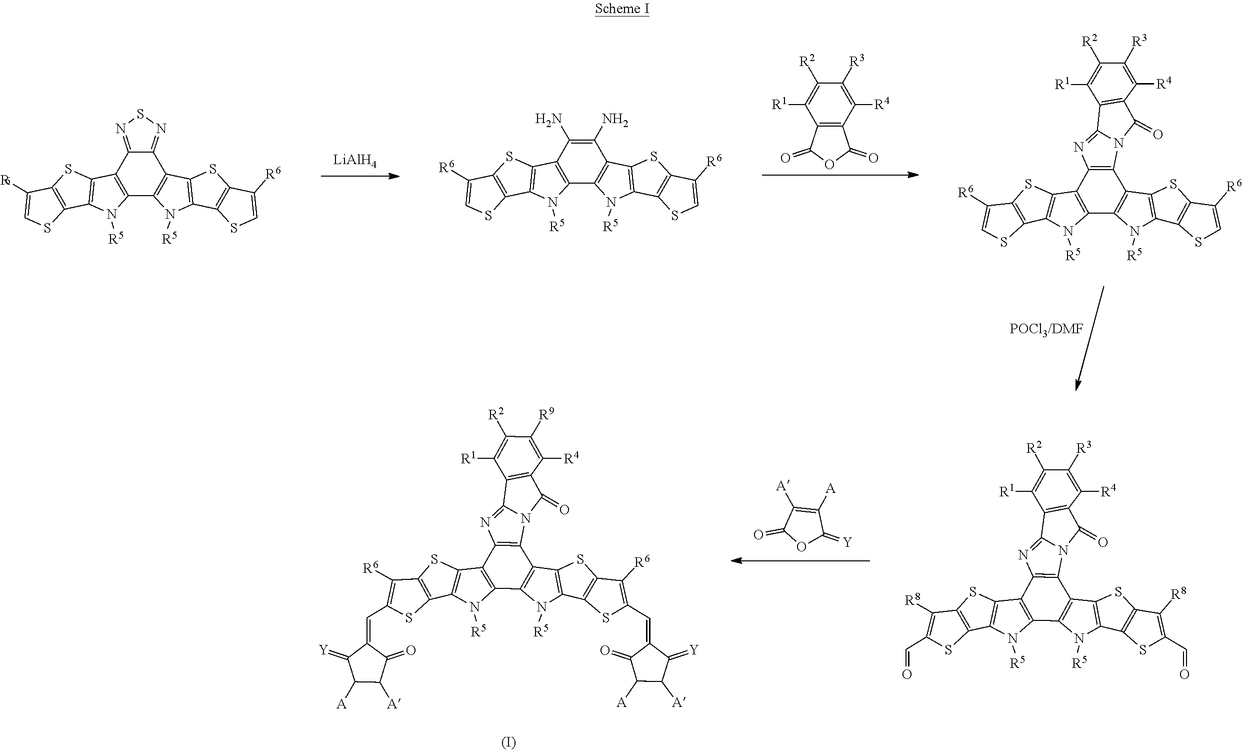

The specific reaction conditions, starting materials and reagents will change depending on the structure of the target compound of Formula I. It should be understood that selection of the specific reaction conditions, starting materials, and reagents used in the synthetic process of Scheme I would be a matter of routine for the skilled person. The starting material used may be a derivative of the precursor used in the synthesis of Y6. Such compounds are commercially available, as are various phthalic hydride derivatives used in the second step of the process of Scheme 1. Similarly, suitable compounds used to introduce functionality in the acceptor end groups are either commercially available or readily derivable from commercially available compounds.

Accordingly, also provided herein is a process for synthesizing the compound of Formula (I) comprising the steps of:
(a) reducing the compound of Formula (VII), optionally with LiAlH₄,

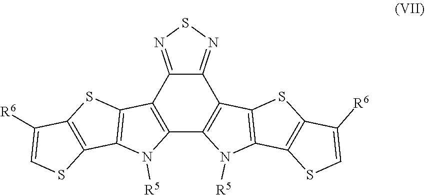

to produce the compound of Formula (VIII)

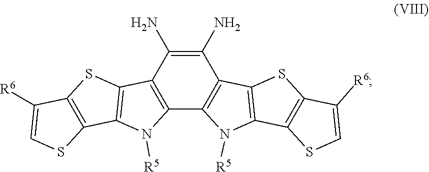

(b) treating the compound of Formula (VIII) with a phthalic hydride of Formula (IX)

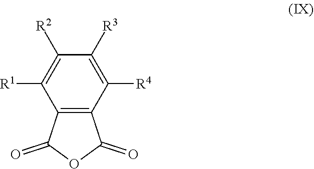

to form a compound of Formula (X)

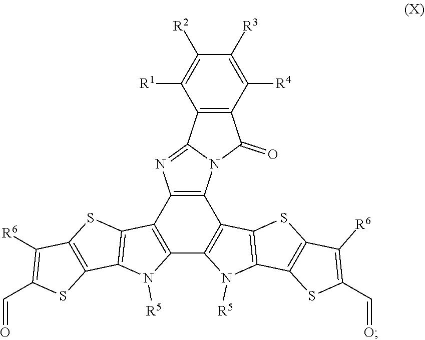

and
(c) reacting the compound of Formula (X) with a compound of Formula (XI) and/or a compound of Formula (XII)

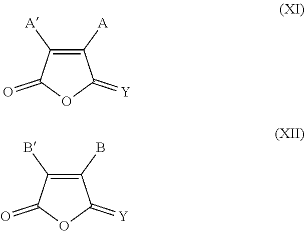

to produce the compound of Formula (I), wherein R¹-R⁶, A, A′, B, B′ and Y are as defined above.

### Semiconductor Materials

The BIID-based NFAs are useful as n-type semiconductors, for example, in bulk heterojunction organic electronic devices.

Bulk heterojunction organic material is made from the combination of one or more BIID-based NFA compound, as described herein, with a donor polymer which has a complementary absorption to the NFA compound. The resulting material is an interpenetrating material in which the BIID-based NFA compounds are intimately mixed, allowing interfaces at appropriate diffusion distance to be dispersed across the active layer. The material is manufactured using standard techniques, to have an appropriate thickness necessary for light absorption in the electronic device.

In one embodiment, a bulk heterojunction blend film can be prepared by dissolving a BIID-based NFA and a donor polymer in an appropriate solvent at different weight ratios, and then casting films by spin-coating. Selection of the appropriate solvent and weight ratios will be dependent on the ultimate application and materials used and their selection is a matter of routine for the skilled person.

The donor polymer used in the manufacture of semiconductor material comprising the BIID-based NFA can be, for example, a middle bandgap donor polymer, such as, but not limited to, PTQ10, J52, and PCDTBT. In a specific embodiment PBDB-T-2F (“PM6”) is employed as a donor polymer used together with a BIID-based NFA in the manufacture of semiconductor material in organic electronic devices. Combination of PM6 with a BIID-based NFA can be used to manufacture bulk heterojunction material as an alternative to Y6-PM6 blends. As demonstrated in the following examples, use of the BIID-based NFA compounds described herein in a blend with PM6 produces bulk semiconductor material with improved properties over Y6-PM6 materials. These examples demonstrate the effectiveness of the present BIID-based NFA as an n-type acceptor.

In another embodiment, the BIID-based NFA can be blended with high-performance p-type materials, such as those described in U.S. Pat. No. 8,927,684, which is incorporated herein by reference in its entirety.

In another embodiment, the semiconductor material comprises one or more BIID-based NFA in a copolymer with other monomers to yield electron-accepting polymers or oligomers. Among organic semiconductors, alternating conjugated polymers of an electron donor (ED) unit and an electron acceptor (EA) unit have attracted more and more attention due to their special properties associated with the donor/acceptor (D/A) structure in the main chain. This D/A structure can effectively lower the band gap of conjugated polymers. Such alternating conjugated polymers can be prepared using one or more BIID-based NFA as the acceptor monomer(s), alone or in combination with one or more additional acceptor monomer(s). BIID-based NFAs, as described herein, can be used as monomers to produce conjugated oligomers or polymers by generally known methods, for example, by Suzuki coupling or Stille coupling.

In one example of this embodiment, the BIID-based NFA monomers are end-capped with Br atoms, and the resulting BIID-based NFA dibromides are then polymerized with aromatic distannyl compounds by a Stille coupling reaction or with aromatic diboronic ester by a Suzuki coupling reaction. These are widely used polymerization methods for the preparation of conjugated polymers that would readily performed by the skilled person. In some embodiments, the BIID-based NFA copolymer or oligomers can be used to fabricate OPVs.

Exemplary groups of co-monomers having electron-donating properties include substituted or unsubstituted phenyls, thienes, fluorenes, carbazoles, benzodithiophenes, pyrroles, indenofluorenes, indolocarbazoles, dibenzosiloles, dithienosiloles, benzo[1,2-b;3,4-b]dithiophenes, benzo[2,1-b:3,4-b′]dithiophenes, cyclopenta[2,1-b:3,4-b]dithiophenes, thieno[3,2-b]thiophenes, thieno[3,4-b]thiophenes and dithieno[3,2-b:2′,3′-d]pyrroles, where any substituents may be one or more of the substituents as defined previously. Specific examples of co-monomers having electron-donating properties include 2,7-bis(4,4,5,5,-tetramethyl-1,3,2-dioxaborolan-2-yl)-9,9-di(2-ethylhexyl)-fluorene, fluorene, carbazole and benzodithiophene.

Some examples of electron-accepting monomers include substituted or unsubstituted benzothiadiazole, thienopyrazine, quinoxaline, dihydropyrrolo[3,4-]pyrrole-1,4-dione, thieno[3,4-b]thiophene, where any substituents may be one or more of the substituents as defined previously.

Electron-accepting monomers may be copolymerized with electron-donating monomers in various ratios to tune the electronic properties of the resulting oligomer or polymer. The ratio of electron-accepting monomer to electron-donating monomer may be in a range of from 1:99 to 99:1 mol %, preferably 40:60 to 60:40 mol %. In oligomers or polymers where other electron-accepting monomers are present, the ratio of BIID-based NFA monomers from to the other electron-accepting monomers is optionally 99:1 to 10:90 mol %.

Oligomers and polymers of the present invention optionally have from 2 to 20,000 monomeric units, or from 10 to 10,000 monomeric units.

Oligomers and polymers of the present invention may be cast as thin films or membranes by methods generally known in the art, for example, spin-coating, casting or printing, which can be used for assembly into organic electronic devices.

Any of the semiconductor materials described herein, comprising one or more BIID-based NFA, can be incorporated in an organic electronic device (e.g., an organic photovoltaic cell). Accordingly, the present application further provides an organic electronic device, comprising the semiconductor material made with a BIID-based NFA and a donor polymer or made using a BIID-based NFA-containing co-polymer. Such organic electronic devices can be, for example, an optoelectronic device, an electroluminescence device, a field effect transistor, an optical sensor, a photovoltaic device (e.g., a solar cell), or a thermoelectric device.

To gain a better understanding of the invention described herein, the following examples are set forth. It should be understood that these examples are for illustrative purposes only. Therefore, they should not limit the scope of this invention in any way.

### EXAMPLES

### Example 1: Synthesis and Use of an NFA Having a 5a,9a-dihydro-11H-benzo[4,5]imidazo[2,1-a]isoindol-11-one (BIID) Core Structure

In the present Example an NFA was synthesized based on the electron-withdrawing core, 5a,9a-dihydro-11H-benzo[4,5]imidazo[2,1-a]isoindol-11-one (Scheme 2). The synthesized BIID-based NFA can be used as n-type semiconductor, for example, in bulk heterojunction solar cells. As a preliminary result, a PCE of 11.2% with a high V_{OC} of 0.95 V was achieved with the synthesized BIID-based NFA when blended with PM6. Compared with the device performance from the benchmark NFA Y6 in the same configuration, the overall PCE is comparable but the V_{OC} has been significantly increased by 17% to 0.95 V (0.81 V for the PM6:Y6 blend). The UV-Vis spectroscopy and cyclic voltammetry study showed both NFAs have almost the same E_{g} and HOMO/LUMO levels, however, the obtained increase in V_{OC} indicates that this BIID-based NFA is able to suppress the recombination losses, which are the main cause of large energy loss in OPVs. Moreover, similar BIID-based NFAs comprising additional solubilizing side chains attached to the BIID core can be synthesized using the same synthetic approach as described in this Example. The solubility of the resulting NFAs can thus be adjusted with relative ease for the printing process.

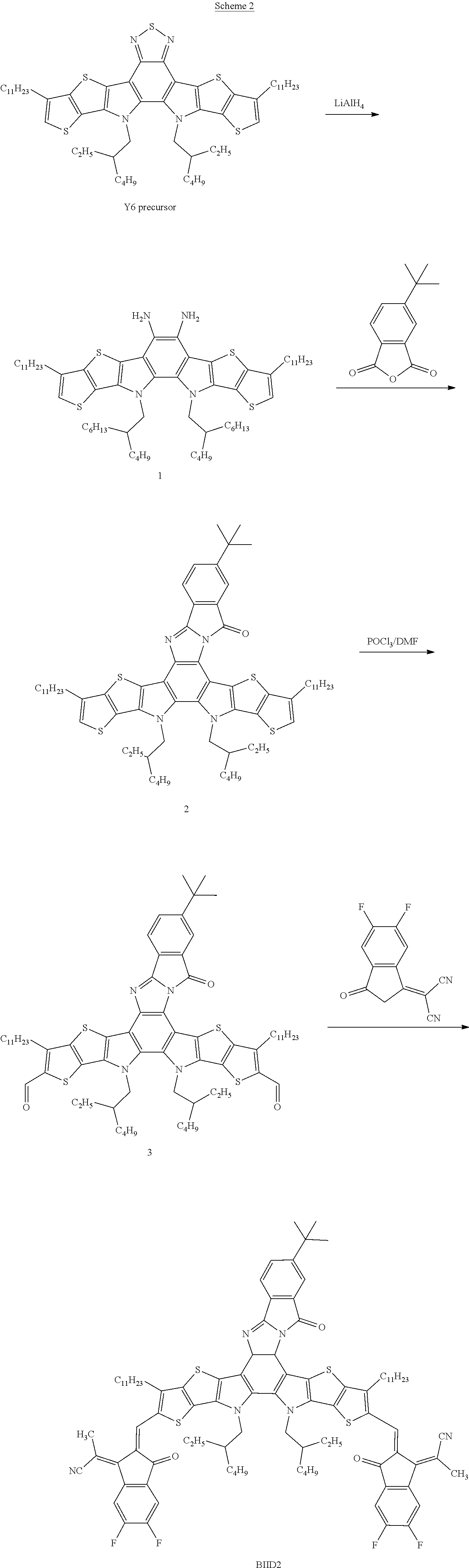

### Synthesis of Compound 2

Y6 precursor and LiAlH₄ were added to an argon protected flask. Then anhydrous THF was added and the reaction mixture was stirred under reflux overnight. Upon cooling to room temperature, the reaction solution was poured into saturated NH₄Cl aqueous solution. Water and ethyl acetate were added for extraction. The organic layer was separated and dried over MgSO₄. After removal of the solvent, crude compound 1 was obtained without further purification. To the flask containing compound 1 AcOH and phthalic anhydride were added and the reaction mixture was stirred under reflux for 6 hrs. Then the solvent was removed and Ac₂O was added. The reaction mixture was then stirred under reflux for 8 hrs. The heating was withdrawn and the solution was let stand still overnight. The precipitate was collected by filtration and washed with methanol. Then the crude product was subject to silica gel column chromatography to give compound 2, which are two separated isomers. ¹H NMR (C₆D6, 600 MHz), δ (ppm): δ 7.63 (d, J=1.8 Hz, 1H), 7.42 (d, J=6.0 Hz, 1H), 6.97 (dd, J=6.0, 1.8 Hz, 1H), 6.70 (s, 1H), 6.67 (s, 1H), 4.86-4.73 (m, 4H), 2.72 (t, J=7.8 Hz, 2H), 2.61 (t, J=7.8 Hz, 2H), 2.21-2.10 (m, 2H), 1.86-1.67 (m, 4H), 1.44-1.15 (m, 34H), 1.02 (s, 9H), 1.00-0.71 (m, 18H), 0.69-0.50 (m, 12H).

### Synthesis of Compound 3

To a solution of 2 in DMF and ClCH₂CH₂Cl, POCl₃ was added slowly at 0° C. under argon after being stirred for 1 h at 0° C., the solution was refluxed overnight. Then it was poured into DI water and extracted with dichloromethane. After removal of the solvent, the crude product was purified by silica gel column chromatography to give compound 3. ¹H NMR (C₆D6, 600 MHz), δ (ppm): δ 10.01 (s, 1H), 9.97 (s, 1H), 7.64 (s, 1H), 7.42 (d, J=7.8 Hz, 1H), 6.99 (dd, J=7.8, 1.8 Hz, 1H), 4.75-4.65 (m, 4H), 2.82 (t, J=7.8 Hz, 2H), 2.72 (t, J=7.8 Hz, 2H), 2.11-2.00 (m, 2H), 1.78-1.60 (m, 4H), 1.42-1.14 (m, 34H), 1.03 (s, 9H), 1.00-0.71 (m, 18H), 0.64-0.48 (m, 12H).

### Synthesis of BIID2

3 and INCN-2F were mixed in a flask. Then chloroform and pyridine were added. (The reaction mixture was stirred under reflux overnight. Then solvent was removed and the crude product was subject to silica gel column chromatography to give BIID2. ¹H NMR (CD₂Cl₂, 600 MHz), δ (ppm): δ 9.13 (s, 2H), 8.56-8.50 (m, 2H), 7.89 (s, 1H), 7.75-7.67 (m, 4H), 4.80-4.70 (m, 4H), 3.30-3.19 (m, 4H), 2.04-1.94 (m, 2H), 1.93-1.83 (m, 4H), 1.43 (s, 9H), 1.42-1.07 (m, 34H), 1.05-0.81 (m, 18H), 0.79-0.57 (m, 12H).

### Optoelectronic Properties of BIID2

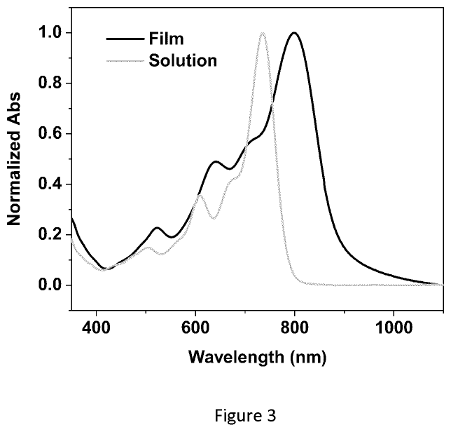

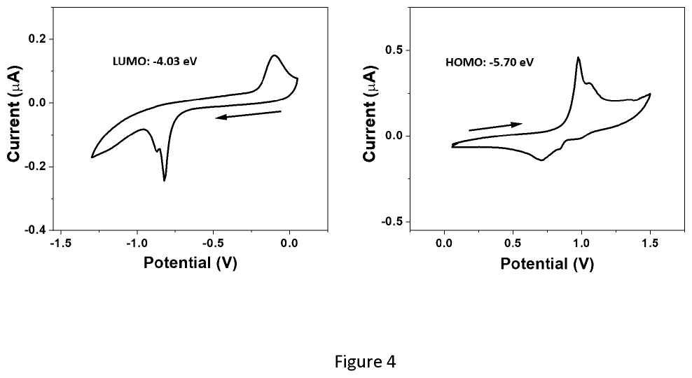

The absorption profile of BIID2 was characterized by UV-Vis absorption spectroscopy. Both the spin-coated film and solution (in chloroform) were measured (FIG. 3). The film absorption showed a significant red-shifted (^{˜}80 nm) spectrum with an absorption maximum at 800 nm, compared to the solution absorption. This result indicates that there exists strong aggregation and n-n interaction in the solid state. Using the film absorption onset, the optical bandgap for this polymer was determined to be around 1.39 eV. The HOMO/LUMO levels of BIID2 were estimated from the onset potentials of oxidative and reductive cyclic voltammograms. Using the redox onsets, the HOMO level was found to be −5.70 eV and the LUMO level to be −4.03 eV. The CV curves are shown in FIG. 4. The CV of Y6 was measured as a comparison and the HOMO/LUMO levels were found to be −5.71/−4.05 eV, which are almost the same as BIID2.

### OPV Performance

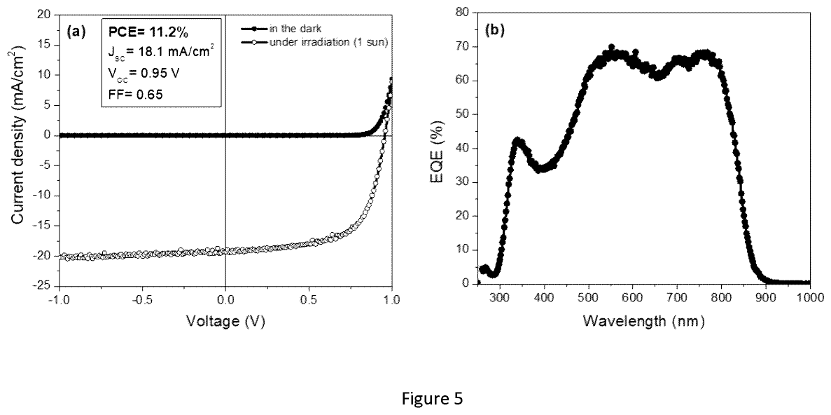

To evaluate the PV performance of BIID2, it was with PM6 as the active layer in an inverted device structure (ZnO as the electron injection layer and MoO₃ as the hole injection layer). The active area was 1 cm². The current-voltage (J-V) characteristics were measured in air under air mass 1.5 global (AM 1.5G) irradiation of 100 mW/cm². The J-V curves and EQE spectrum of the fabricated solar cell are shown in FIG. 5. The OPV device showed a PCE of 11.2% with a high V_{oc} of 0.95 V, J_{sc} of 18.1 mA/cm² and high FF of 0.65, demonstrating the value of BIID2 as an effective PV acceptor providing a PCE similar to that of Y6, but with improved V_{oc}.

### Example 2: A Non-Fullerene Acceptor Based on a Two-Dimensional Electron-Deficient Core for Organic Photovoltaic Cells

In this Example, the BIID core structure was as the basis for an acceptor molecule with increased optical bandgap, in comparison to Y6, and to finely tuned energy levels to increase the V_{oc} of each individual cell. Specifically, the BIID core structure was modified by extending the centre electron-deficient core in the y-direction and changing the C₈-alkyl chain to C₁₂-alkyl chain, to synthesize a new two-dimensional NFA: 2,2′-((2Z,2′Z)-((16,17-bis(2-butyloctyl)-10-oxo-3,13-diundecyl-4c,11a,16,17-tetrahydro-10H-isoindolo[2′,1′:1,2]imidazo[4,5-e]thieno[2″,3″:4′,5′]thieno[2′,3′:4,5]pyrrolo[3,2-g]thieno[2′,3′:4,5]thieno[3,2-b]indole-2,14-diyl)bis(methanylylidene))bis(5,6-difluoro-3-oxo-2,3-dihydro-1H-indene-2,1-diylidene))dimalononitrile (BIID3).

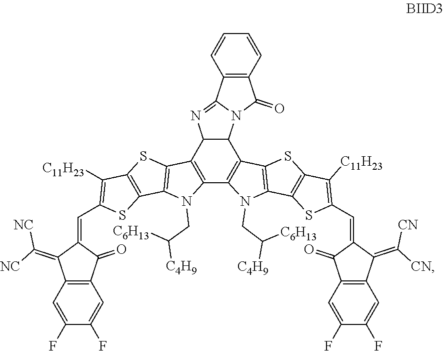

BIID3 has an optical bandgap of 1.38 eV, which is slightly larger than that of Y6 (1.31 eV). It is interesting to point out that cyclic voltammetry measurements show that BIID3 has HOMO/LUMO levels of −5.68/−4.04 eV in the thin film state, which are similar to those of Y6 (5.71/−4.05 eV). BIID3 was tested with the donor polymer PM6 in a 1 cm² inverted OPV device and achieved high PCE of 13.7% under one sun irradiation and 19.4% under an indoor LED illumination.

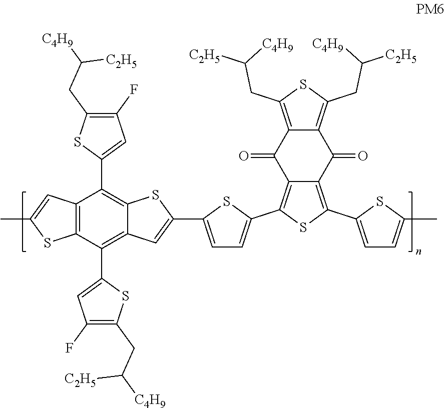

The overall synthesis of BIID3 consisted of three major steps (Scheme 3).

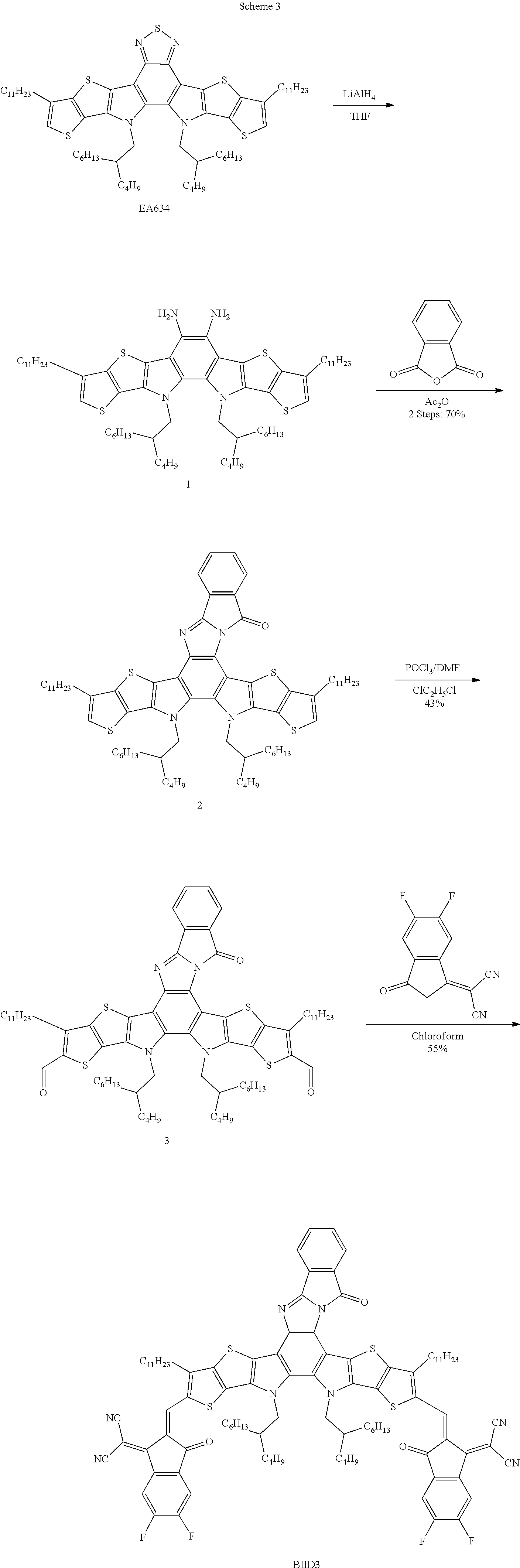

First, the starting material EA634 was reduced and then reacted with phthalic anhydride to yield 2. Then the aldehyde functional groups were introduced by treating 2 with POCl₃ and DMF to generate 3. Finally, the condensation of 3 and INCN-2F gave the product BIID3.

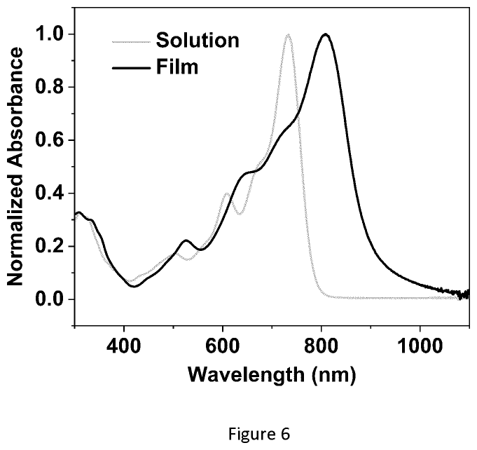

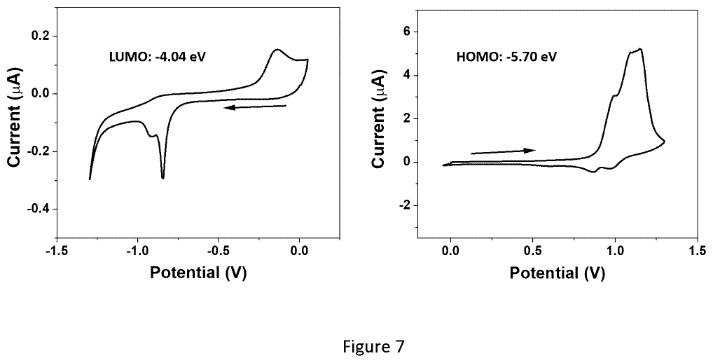

The thermal stability of BIID3 was characterized by by Thermogravimetric Analysis (TGA). An onset decomposition temperature of 300° C. corresponding to a 1% weight loss was found. The absorption profile of BIID was characterized by UV-Vis absorption spectroscopy. Both the spin-coated film and solution (in chloroform) were measured (FIG. 6). The film absorption spectrum showed a significant red-shifted of 75 nm with an absorption maximum at 808 nm, compared to the solution absorption. This result indicates that there exists strong aggregation and n-n interaction in the solid state. Using the film absorption onset, we determined that the optical bandgap for this polymer is around 1.38 eV. The HOMO/LUMO levels of BIID3 were estimated from the onset potentials of oxidative and reductive cyclic voltammograms. Using the redox onsets, the HOMO level was found to be −5.68 eV and the LUMO level to be −4.04 eV. The CV curves are shown in FIG. 7. The CV of Y6 was measured under the same conditions for comparison and it was found that the HOMO/LUMO levels are −5.71/−4.05 eV, which are almost the same as those of BIID3.

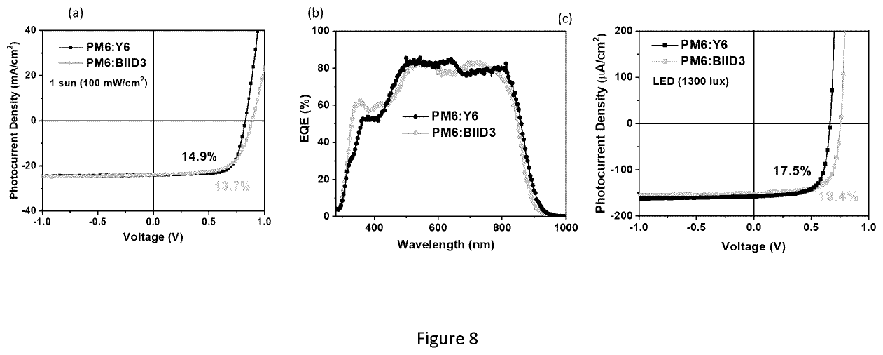

To evaluate the photovoltaic performance of BIID3, it was blending with PM6 as the active layer in an inverted OPV device structure (ZnO as the electron extraction layer and MoO₃ as the hole extraction layer). The active area was 1 cm². A comparison device was fabricated using Y6:PM6 as the active layer and evaluated under the same conditions for comparison. The devices were first investigated under AM 1.5 G irradiation of 100 mW/cm² in the air. The J-V curves and EQE spectra of the fabricated OPV devices are shown in FIG. 8. The device based on BIID3 showed a PCE of 13.7% with a V_{OC} of 0.89 V, J_{SC} of 23.8 mA/cm² and an FF of 0.64, while the device based on Y6 showed a PCE of 14.5% with a V_{OC} of 0.82 V, J_{SC} of 24.6 mA/cm² and an FF of 0.72. Without wishing to be bound by theory, it appears that the PCE difference is mainly caused by the difference in FFs in these two devices.

The charge mobilities of these two devices were also measured. The BIID3 device had an electron mobility of 8.73×10⁻⁵ cm²/V s, and Y6 had an electron mobility of 9.66×10⁻⁵ cm²/V s. The EQE spectra showed that there is blue shift for the BIID3 containing device, which is partially responsible for the slightly smaller current density.

Following previous studies on indoor OPVs,³²⁻³⁴ the photovoltaic performance of the OPV devices were measured under different LED light intensities from 56 lux to 1300 lux (Tables 2 and 4). As shown in these Tables, the PCEs of the OPV devices increase as the light intensity increases in this light intensity range. This is because the increased carrier density at a higher light intensity reduces the effect of leakage current and trap-assisted recombination. At an illumination of 1300 lux, the BIID3-based device showed a PCE of 19.4% with a V_{OC} of 0.75 V, J_{SC} of 153 μA/cm² and an FF of 0.70 while the Y6-based device showed a PCE of 17.5% with a V_{OC} of 0.66 V, J_{SC} of 157 μA/cm² and an FF of 0.70 (FIG. 8c). These results demonstrated that the BIID3 device has a significantly higher V_{OC} than the Y6 device, which makes it more suitable for indoor light harvesting to power electronic devices for applications in Internet of things (IoT) than a Y6-based device.

TABLE 2
Device Parameters for the BIID3 Device

| LED light | LED light | | | | |
|---|---|---|---|---|---|
| intensity | intensity | | | | |
| (Lux) | (μW/cm²) | PCE (%) | J_{SC} (μA/cm²) | V_{OC} (V) | FF |
| 1300 | 412 | 19.4 | 153.0 | 0.75 | 0.70 |
| 768 | 241 | 19.0 | 86.7 | 0.73 | 0.73 |
| 505 | 159 | 18.7 | 58.1 | 0.72 | 0.71 |
| 409 | 128 | 18.6 | 47.3 | 0.71 | 0.71 |
| 334 | 105 | 18.4 | 38.9 | 0.70 | 0.71 |
| 201 | 63 | 18.1 | 23.5 | 0.68 | 0.71 |
| 100 | 32 | 17.2 | 11.8 | 0.66 | 0.70 |
| 56 | 18 | 16.6 | 6.7 | 0.64 | 0.70 |

TABLE 3
Device Parameters for the Y6 Device

| | LED light | | | | |
|---|---|---|---|---|---|
| LED light | intensity | | J_{SC} | | |
| intensity (Lux) | (μW/cm²) | PCE (%) | (μA/cm²) | V_{OC} (V) | FF |
| 1311 | 414 | 17.5 | 157.3 | 0.66 | 0.70 |
| 768 | 241 | 17.0 | 91.1 | 0.64 | 0.70 |
| 501 | 158 | 16.6 | 60.5 | 0.62 | 0.70 |
| 407 | 128 | 16.4 | 49.2 | 0.61 | 0.70 |
| 266 | 83 | 16.1 | 32.3 | 0.60 | 0.69 |
| 202 | 64 | 15.5 | 24.6 | 0.59 | 0.68 |
| 101 | 32 | 14.9 | 12.4 | 0.56 | 0.69 |
| 57 | 18 | 14.5 | 7.0 | 0.53 | 0.68 |

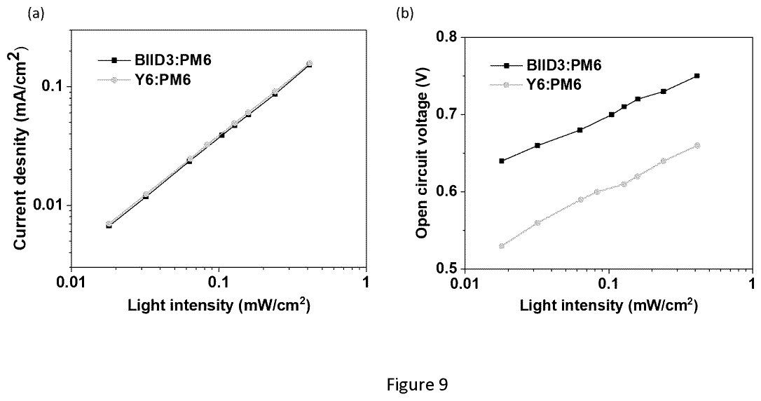

The dependence of J_{SC} on the light intensity was evaluated in FIG. 9a. The fitting slopes of J_{SC} versus light intensity are 0.995 and 0.991 for BIID:PM6 and Y6:PM6, respectively, indicating the negligible bimolecular recombination in both systems and constant high photon-to-electron conversion ratios with the decrease of light intensity. Previous studies have demonstrated that the trap-assisted recombination has significant impacts on the V_{OC} of indoor OPV devices since the charge-carrier density is extremely low under indoor conditions²⁷. The dependence of V_{OC} on light intensities of these two blends was measured at different light intensities (FIG. 9b). The ideal factors, n, calculated from the slope of the graph, were 1.37 and 1.56 for BIID3:PM6 and Y6:PM6, respectively. These results indicate BIID3:PM6 has a lower extent of trap-assisted recombination. Without wishing to be bound by theory, this could be a reason that BIID3 offers a much higher V_{OC} than Y6, although they have similar frontier energy levels.

### CONCLUSIONS

The present example describes the synthesis of a BIID-based NFA using the two-dimensional rigid fused electron deficient BIID core. This compound has a larger n-conjugation than Y6 and includes versatile functional groups that are suitable for chemical modification in order to further optimize the NFA for different applications.

The OPV device based on BIID3:PM6 showed a decent PCE of 13.7% under the one sun irradiation and a high PCE of 19.4% under the LED illumination. This good performance of the BIID3 in OPVs is credited to its high V_{OC}, which benefits mainly from the suppressed trap-assisted recombination. The BIID core-based molecular design allows further electronic property tuning, precise morphology optimization, and solution processability for the use in next-step high performance indoor light OPVs.

### REFERENCES

(1) Yu, G.; Gao, J.; Hummelen, J. C.; Wudl, F.; Heeger, A. J. Polymer Photovoltaic Cells: Enhanced Efficiencies via a Network of Internal Donor-Acceptor Heterojunctions. Science (80-.). 1995, 270 (5243), 1789-1791. https://doi.org/10.1126/science.270.5243.1789.
(2) Kaltenbrunner, M.; White, M. S.; Glowacki, E. D.; Sekitani, T.; Someya, T.; Sariciftci, N. S.; Bauer, S. Ultrathin and Lightweight Organic Solar Cells with High Flexibility. Nat. Commun. 2012, 3 (1), 770. https://doi.org/10.1038/ncomms1772.
(3) Li, G.; Zhu, R.; Yang, Y. Polymer Solar Cells. Nat. Photonics 2012, 6 (3), 153-161. https://doi.org/10.1038/nphoton.2012.11.
(4) Wang, G.; Melkonyan, F. S.; Facchetti, A.; Marks, T. J. All-Polymer Solar Cells: Recent Progress, Challenges, and Prospects. Angew. Chemie—Int. Ed. 2019, 4129-4142. https://doi.org/10.1002/anie.201808976.
(5) Yan, C.; Barlow, S.; Wang, Z.; Yan, H.; Jen, A. K.-Y.; Marder, S. R.; Zhan, X. Non-Fullerene Acceptors for Organic Solar Cells. Nat. Rev. Mater. 2018, 3 (3), 18003. https://doi.org/10.1038/natrevmats.2018.3.
(6) Cui, Y.; Yao, H.; Zhang, J.; Xian, K.; Zhang, T.; Hong, L.; Wang, Y.; Xu, Y.; Ma, K.; An, C.; He, C.; Wei, Z.; Gao, F.; Hou, J. Single-Junction Organic Photovoltaic Cells with Approaching 18% Efficiency. Adv. Mater. 2020, 32 (19), 1908205. https://doi.org/10.1002/adma.201908205.
(7) Liu, Q.; Jiang, Y.; Jin, K.; Qin, J.; Xu, J.; Li, W.; Xiong, J.; Liu, J.; Xiao, Z.; Sun, K.; Yang, S.; Zhang, X.; Ding, L. 18% Efficiency Organic Solar Cells. Sci. Bull. 2020, 65 (4), 272-275. https://doi.org/10.1016/j.scib.2020.01.001.
(8) Liu, S.; Yuan, J.; Deng, W.; Luo, M.; Xie, Y.; Liang, Q.; Zou, Y.; He, Z.; Wu, H.; Cao, Y. High-Efficiency Organic Solar Cells with Low Non-Radiative Recombination Loss and Low Energetic Disorder. Nat. Photonics 2020, 14 (5), 300-305. https://doi.org/10.1038/s41566-019-0573-5.
(9) Xu, X.; Feng, K.; Lee, Y. W.; Woo, H. Y.; Zhang, G.; Peng, Q. Subtle Polymer Donor and Molecular Acceptor Design Enable Efficient Polymer Solar Cells with a Very Small Energy Loss. Adv. Funct. Mater. 2020, 1907570, 1-9. https://doi.org/10.1002/adfm.201907570.
(10) Zhou, Z.; Liu, W.; Zhou, G.; Zhang, M.; Qian, D.; Zhang, J.; Chen, S.; Xu, S.; Yang, C.; Gao, F.; Zhu, H.; Liu, F.; Zhu, X. Subtle Molecular Tailoring Induces Significant Morphology Optimization Enabling over 16% Efficiency Organic Solar Cells with Efficient Charge Generation. Adv. Mater. 2020, 32 (4), 1-8. https://doi.org/10.1002/adma.201906324.
(11) Fan, B.; Zeng, Z.; Zhong, W.; Ying, L.; Zhang, D.; Li, M.; Peng, F.; Li, N.; Huang, F.; Cao, Y. Optimizing Microstructure Morphology and Reducing Electronic Losses in 1 Cm2 Polymer Solar Cells to Achieve Efficiency over 15%. ACS Energy Lett. 2019, 4 (10), 2466-2472. https://doi.org/10.1021/acsenergylett.9b01447.
(12) Yan, T.; Song, W.; Huang, J.; Peng, R.; Huang, L.; Ge, Z. 16.67% Rigid and 14.06% Flexible Organic Solar Cells Enabled by Ternary Heterojunction Strategy. Adv. Mater. 2019, 31 (39), 1902210. https://doi.org/https://doi.org/10.1002/adma.201902210.
(13) Kniepert, J.; Paulke, A.; Perdigón-Toro, L.; Kurpiers, J.; Zhang, H.; Gao, F.; Yuan, J.; Zou, Y.; Le Corre, V. M.; Koster, L. J. A.; Neher, D. Reliability of Charge Carrier Recombination Data Determined with Charge Extraction Methods. J. Appl. Phys. 2019, 126 (20), 205501. https://doi.org/10.1063/1.5129037.
(14) Zhao, W.; Li, S.; Yao, H.; Zhang, S.; Zhang, Y.; Yang, B.; Hou, J. Molecular Optimization Enables over 13% Efficiency in Organic Solar Cells. J. Am. Chem. Soc. 2017, 139 (21), 7148-7151. https://doi.org/10.1021/jacs.7b02677.
(15) Yuan, J.; Zhang, Y.; Zhou, L.; Zhang, G.; Yip, H. L.; Lau, T. K.; Lu, X.; Zhu, C.; Peng, H.; Johnson, P. A.; Leclerc, M.; Cao, Y.; Ulanski, J.; Li, Y.; Zou, Y. Single-Junction Organic Solar Cell with over 15% Efficiency Using Fused-Ring Acceptor with Electron-Deficient Core. Joule 2019, 3 (4), 1140-1151. https://doi.org/10.1016/j.joule.2019.01.004.
(16) Yuan, J.; Zhang, Y.; Zhou, L.; Zhang, C.; Lau, T.; Zhang, G.; Lu, X.; Yip, H.; So, S. K.; Beaupré, S.; Mainville, M.; Johnson, P. A.; Leclerc, M.; Chen, H.; Peng, H.; Li, Y.; Zou, Y. Fused Benzothiadiazole: A Building Block for N-Type Organic Acceptor to Achieve High-Performance Organic Solar Cells. Adv. Mater. 2019, 31 (17), 1807577. https://doi.org/10.1002/adma.201807577.
(17) Yoo, J. J.; Seo, G.; Chua, M. R.; Park, T. G.; Lu, Y.; Rotermund, F.; Kim, Y. K.; Moon, C. S.; Jeon, N. J.; Correa-Baena, J. P.; et al. Efficient Perovskite Solar Cells via Improved Carrier Management. Nature 2021, 590 (7847), 587-593. https://doi.org/10.1038/s41586-021-03285-w.
(18) Vandewal, K.; Tvingstedt, K.; Gadisa, A.; Inganäs, O.; Manca, J. V. On the Origin of the Open-Circuit Voltage of Polymer-Fullerene Solar Cells. Nat. Mater. 2009, 8 (11), 904-909. https://doi.org/10.1038/nmat2548.
(19) Elumalai, N. K.; Uddin, A. Open Circuit Voltage of Organic Solar Cells: An in-Depth Review. Energy Environ. Sci. 2016, 9 (2), 391-410. https://doi.org/10.1039/C5EE02871J.
(20) Bakulin, A. A.; Rao, A.; Pavelyev, V. G.; van Loosdrecht, P. H. M.; Pshenichnikov, M. S.; Niedzialek, D.; Cornil, J.; Beljonne, D.; Friend, R. H. The Role of Driving Energy and Delocalized States for Charge Separation in Organic Semiconductors. Science (80-.). 2012, 335 (6074), 1340-1344. https://doi.org/10.1126/science.1217745.
(21) Yao, J.; Kirchartz, T.; Vezie, M. S.; Faist, M. A.; Gong, W.; He, Z.; Wu, H.; Troughton, J.; Watson, T.; Bryant, D.; et al. Quantifying Losses in Open-Circuit Voltage in Solution-Processable Solar Cells. Phys. Rev. Appl. 2015, 4 (1), 14020. https://doi.org/10.1103/PhysRevApplied.4.014020.
(22) Qian, D.; Zheng, Z.; Yao, H.; Tress, W.; Hopper, T. R.; Chen, S.; Li, S.; Liu, J.; Chen, S.; Zhang, J.; et al. Design Rules for Minimizing Voltage Losses in High-Efficiency Organic Solar Cells. Nat. Mater. 2018, 17 (8), 703-709. https://doi.org/10.1038/s41563-018-0128-z.
(23) Liu, J.; Chen, S.; Qian, D.; Gautam, B.; Yang, G.; Zhao, J.; Bergqvist, J.; Zhang, F.; Ma, W.; Ade, H.; et al. Fast Charge Separation in a Non-Fullerene Organic Solar Cell with a Small Driving Force. Nat. Energy 2016, 1 (7), 16089. https://doi.org/10.1038/nenergy.2016.89.
(24) Lee, H. K. H.; Wu, J.; Barbé, J.; Jain, S. M.; Wood, S.; Speller, E. M.; Li, Z.; Castro, F. A.; Durrant, J. R.; Tsoi, W. C. Organic Photovoltaic Cells-Promising Indoor Light Harvesters for Self-Sustainable Electronics. J. Mater. Chem. A 2018, 6 (14), 5618-5626. https://doi.org/10.1039/c7ta10875c.
(25) Mathews, I.; Kantareddy, S. N.; Buonassisi, T.; Peters, I. M. Technology and Market Perspective for Indoor Photovoltaic Cells. Joule 2019, 3 (6), 1415-1426. https://doi.org/10.1016/j.joule.2019.03.026.
(26) Chen, F. C. Emerging Organic and Organic/Inorganic Hybrid Photovoltaic Devices for Specialty Applications: Low-Level-Lighting Energy Conversion and Biomedical Treatment. Adv. Opt. Mater. 2019, 7 (1), 1-24. https://doi.org/10.1002/adom.201800662.
(27) Ma, L. K.; Chen, Y.; Chow, P. C. Y.; Zhang, G.; Huang, J.; Ma, C.; Zhang, J.; Yin, H.; Hong Cheung, A. M.; Wong, K. S.; So, S. K.; Yan, H. High-Efficiency Indoor Organic Photovoltaics with a Band-Aligned Interlayer. Joule 2020, 4 (7), 1486-1500. https://doi.org/10.1016/j.joule.2020.05.010.
(28) Fan, B.; Du, X.; Liu, F.; Zhong, W.; Ying, L.; Xie, R.; Tang, X.; An, K.; Xin, J.; Li, N.; Ma, W.; Brabec, C. J.; Huang, F.; Cao, Y. Fine-Tuning of the Chemical Structure of Photoactive Materials for Highly Efficient Organic Photovoltaics. Nat. Energy 2018, 3 (12), 1051-1058. https://doi.org/10.1038/s41560-018-0263-4.
(29) Zhao, J.; Li, Y.; Yang, G.; Jiang, K.; Lin, H.; Ade, H.; Ma, W.; Yan, H. Efficient Organic Solar Cells Processed from Hydrocarbon Solvents. Nat. Energy 2016, 1 (2), 15027. https://doi.org/10.1038/nenergy.2015.27.
(30) Ma, L.-K.; Chen, Y.; Chow, P. C. Y.; Zhang, G.; Huang, J.; Ma, C.; Zhang, J.; Yin, H.; Hong Cheung, A. M.; Wong, K. S.; So, S. K.; Yan, H. High-Efficiency Indoor Organic Photovoltaics with a Band-Aligned Interlayer. Joule 2020, 4 (7), 1486-1500. https://doi.org/https://doi.org/10.1016/j.joule.2020.05.010.
(31) Minnaert, B.; Veelaert, P. Efficiency Simulations of Thin Film Chalcogenide Photovoltaic Cells for Different Indoor Lighting Conditions. Thin Solid Films 2011, 519 (21), 7537-7540. https://doi.org/https://doi.org/10.1016/j.tsf.2011.01.362.
(32) Freitag, M.; Teuscher, J.; Saygili, Y.; Zhang, X.; Giordano, F.; Liska, P.; Hua, J.; Zakeeruddin, S. M.; Moser, J.-E.; Grstzel, M.; Hagfeldt, A. Dye-Sensitized Solar Cells for Efficient Power Generation under Ambient Lighting. Nat. Photonics 2017, 11 (6), 372-378. https://doi.org/10.1038/nphoton.2017.60.
(33) Cui, Y.; Wang, Y.; Bergqvist, J.; Yao, H.; Xu, Y.; Gao, B.; Yang, C.; Zhang, S.; Inganäs, O.; Gao, F.; Hou, J. Wide-Gap Non-Fullerene Acceptor Enabling High-Performance Organic Photovoltaic Cells for Indoor Applications. Nat. Energy 2019, 4 (9), 768-775. https://doi.org/10.1038/s41560-019-0448-5.
(34) Bai, F.; Zhang, J.; Zeng, A.; Zhao, H.; Duan, K.; Yu, H.; Cheng, K.; Chai, G.; Chen, Y.; Liang, J.; Ma, W.; Yan, H. A Highly Crystalline Non-Fullerene Acceptor Enabling Efficient Indoor Organic Photovoltaics with High EQE and Fill Factor. Joule 2021, 5 (5), 1231-1245. https://doi.org/10.1016/j.joule.2021.03.020.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A compound of Formula I. 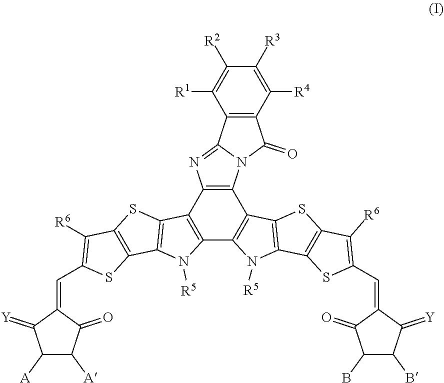
wherein:
R¹, R², R³ and R⁴ are the same or different and each is independently F, H, Cl, Br, I, a substituted or unsubstituted C₁-C₁₂-alkyl, a substituted or unsubstituted C₁-C₁₂-alkoxy, a substituted or unsubstituted aryl group or a substituted or unsubstituted heteroaryl group;
each R⁵ is independently a substituted or unsubstituted C₁-C₃₀-alkyl group;
each R⁶ is independently a substituted or unsubstituted C₁-C₃₀-alkyl group;
each Y is independently C(CN)₂ or O;
A and A′ together with the carbons to which they are attached form an aromatic or heteraromatic ring; and
B and B′ together with the carbons to which they are attached form an aromatic or heteraromatic ring.

2. The compound of claim 1, wherein A and A′ together with the carbons to which they are attached form an aromatic or heteraromatic moiety that is: 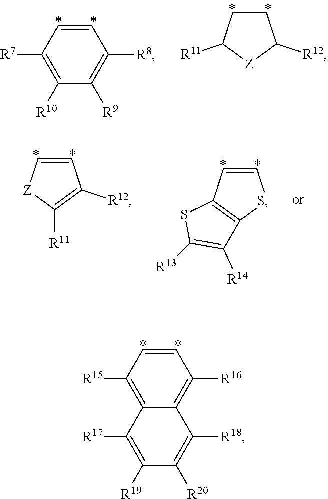
where the asterisks show the point of attachment; and/or B and B′ together with the carbons to which they are attached form an aromatic or heteraromatic ring, for example, B and B′ together with the carbons to which they are attached form an aromatic or heteraromatic moiety that is: 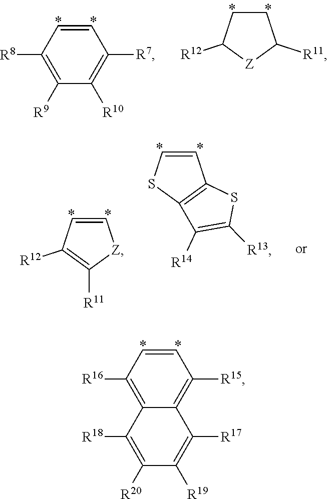
where the asterisks show the point of attachment;
wherein:
R⁷, R⁸, R⁹ and R¹⁰ are each the same or different and are independently F, H, Cl, Br, I, a C₁-C₁₂-alkyl or a C₁-C₁₂-alkoxy group;
Z is O, S, Se, or NR, where R is a C₁-C₁₂-alkyl; and
R¹¹ to R²⁰ are each the same or different and are independently F, H, Cl, Br, I, a C₁-C₁₂-alkyl or a C₁-C₁₂-alkoxy group.

3. The compound of claim 1, wherein Y is C(CN)₂.

4. The compound of claim 1, wherein R⁵ is a branched C₆-C₂₀ alkyl, optionally a branched C₈-alkyl or a branched C₁₂ alkyl.

5. The compound of claim 1, wherein R⁶ is a C₆-C₂₀ alkyl, such as a linear C₁₁ alkyl.

6. The compound of claim 1, wherein one of R¹-R⁴ is a C₁-C₆ alkyl, such as a methyl, ethyl, propyl, or butyl, and the other three are H.

7. The compound of claim 6, wherein R³ is a t-butyl.

8. The compound of claim 1, having the structure of Formula (II), Formula (III), Formula (IV), Formula (V) or Formula (VI):

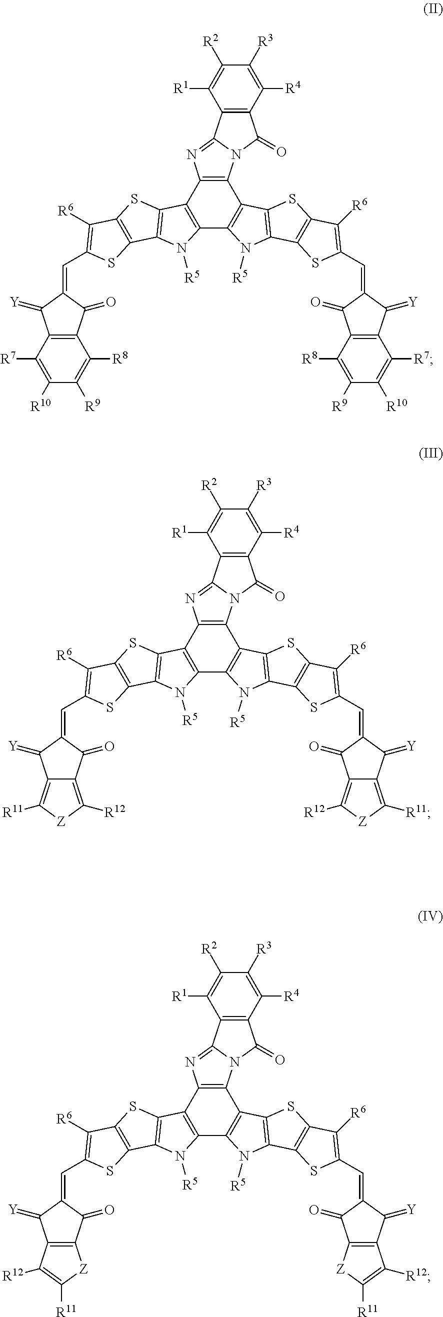

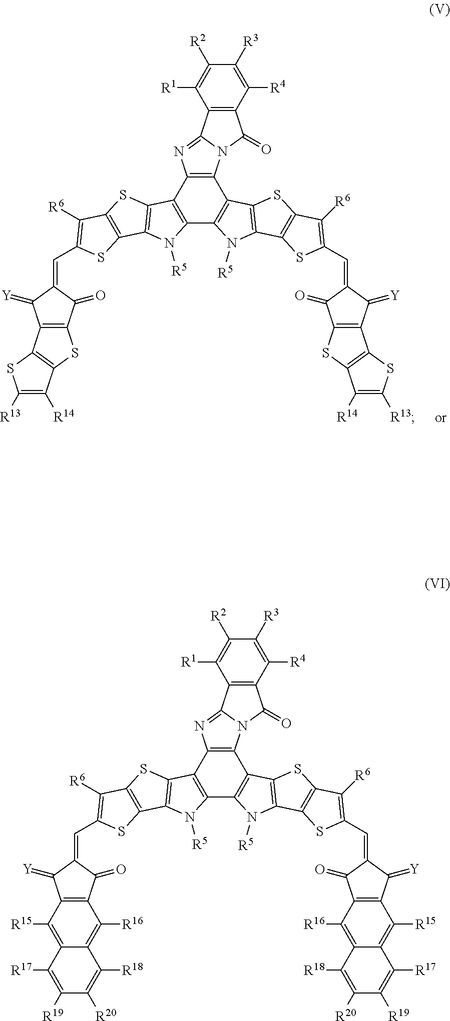

9. The compound of claim 8, which is a compound of Formula II, wherein R7 and R8 are both H and R9 and R10 are each independently H, Cl or F.

10. The compound of claim 9, where each R9 and R10 are the same and are either Cl or F, preferably F.

11. A semiconductor material comprising the compound according to claim 1 in combination with an electron donor polymer.

12. The semiconductor material according to claim 11, wherein the polymer donor is a middle bandgap donor polymer, such as PM6.

13. The semiconductor material according to claim 11, which is a bulk heterojunction organic material.

14. The semiconductor material according to claim 11, wherein the semiconductor material is a component in an organic electronic device, such as an optoelectronic device, an electroluminescence device, a field effect transistor, an optical sensor, a photovoltaic device, or a thermoelectric device.

15. A polymer or an oligomer comprising the compound according to claim 1 copolymerized with an electron-donating comonomer or an electron-withdrawing co-monomer.

16. The polymer or oligomer according to claim 15 having a ratio of electron-accepting monomer to electron-donating or electron-withdrawing co-monomer in a range of from 1:99 to 99:1 mol %.

17. The polymer or oligomer according to claim 15, comprising from 2 to 20,000 monomeric units.

18. The polymer or oligomer according to claim 15, comprising the electron-donating co-monomer, which is one or more of a substituted or unsubstituted phenyl, thiophene, fluorene, carbazole, benzodithiophene, pyrrole, indenofluorene, indolocarbazole, dibenzosilole, dithienosilole, benzo[1,2-b;3,4-b]dithiophene, benzo[2,1-b:3,4-b′]dithiophene, cyclopenta[2,1-b:3,4-b′]dithiophene, thieno[3,2-b]thiophene, thieno[3,4-b]thiophene or dithieno[3,2-b:2′,3′-d]pyrrole, where the substituent, if present, is one or more of F, a C₁-C₃₀-alkyl, C₁-C₃₀-alkenyl, C₁-C₃₀-alkynyl, a C₅-C₃₀-aryl group, or a C₃-C₃₀-heteroaryl group having one or more of N, O or S in the ring.

19. The polymer or oligomer according to claim 15, comprising the electron-withdrawing co-monomer, which is one or more of 2,1,3-benzothiadiazole, 2H-benzo[d][1,2,3]triazole, benzo[c][1,2,5]oxadiazole, benzo[c][1,2,5]selenadiazole, diketopyrrolo[3,4-c]pyrrole-1,4-dione, ester or ketone substituted thieno[3,4-b]thiophene, thieno[3,4-c]pyrrole-4,6-dione, isoindigo, or quinoxaline, where the substituent, if present, is one or more of F, a C₁-C₃₀-alkyl, C₁-C₃₀-alkenyl, C₁-C₃₀-alkynyl, a C₆-C₃₀-aryl group or a C₃-C₃₀-heteroaryl group having one or more of N, O or S in the ring.

20. A film or membrane comprising the compound of claim 1.

21. An optoelectronic device comprising the semiconductor material of claim 11.

22. The optoelectronic device according to claim 21, which is an electroluminescence device, a field effect transistor, an optical sensor, a photovoltaic device, or a thermoelectric device.

23. A process for synthesizing a compound of Formula (I), as defined in claim 1, comprising the steps of:
a) reducing the compound of Formula (VII), optionally with LiAlH4, 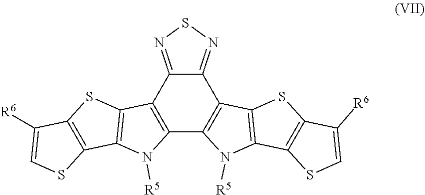
to produce the compound of Formula (VIII) 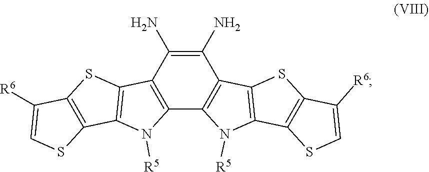
b) treating the compound of Formula (VIII) with a phthalic hydride of Formula (IX) 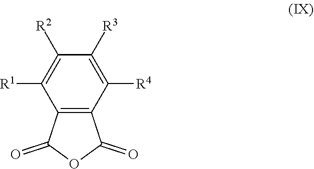
to form a compound of Formula (X) 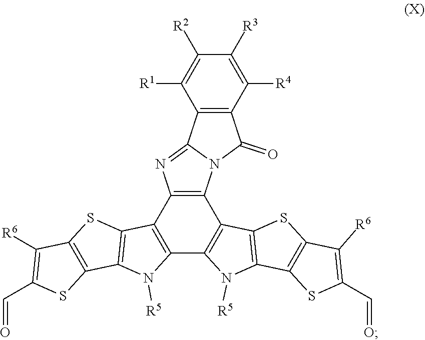
and
c) reacting the compound of Formula (X) with a compound of Formula (XI) and/or a compound of Formula (XII) 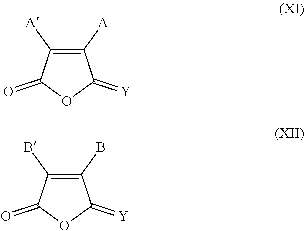
to produce the compound of Formula (I), wherein R¹-R⁶, A, A′, B, B′ and Y are as defined in claim 1.
